(19) United States
(12) Patent Application Publication
(10) Pub. No.: US 2011/0008476 A1
(43) Pub. Date: Jan. 13, 2011

(54) Methods of Treating or Preventing Inflammatory Diseases of the Intestinal Tract

(75) Inventors: Finn Erik Johansen, Oslo (NO); Anders Sandvik, Oslo (NO); Rolf Einar Engstad, Tromson (NO)

Correspondence Address: ROTHWELL, FIGG, ERNST & MANBECK, P.C., 1425 K STREET, N.W., SUITE 800, WASHINGTON, DC, 20005, US

(73) Assignee: BIOTEC PHARMACON ASA, Tromsø (NO)

(21) Appl. No.: 12/742,543

(22) PCT Filed: Nov. 13, 2008
(86) PCT No.: PCT/GB2008/003850

§ 371 Date: Sep. 24, 2010

(30) Foreign Application Priority Data
Nov. 13, 2007 (GB) 0722283.9
May 22, 2008 (GB) 0809339.5
Oct. 16, 2008 (GB) 0819010.0

(51) Int. Cl. A61K 36/28; A61K 31/715; A61K 38/00; A61P 1/00

(52) U.S. Cl. 424/764; 514/54; 514/1.1

(57) ABSTRACT

The present invention relates to a glucan derived from yeast having a beta-(1, 3)-backbone with one or more beta-(1, 3)-side chains linked thereto for use in the treatment or prevention of inflammatory bowel disease and related diseases of abnormal bowel function in an animal, in particular to such uses employing a soluble glucan, e.g. from Saccharomyces cerevisiae, preferably when administered orally. The invention also relates to alternative treatments of inflammatory bowel disease and related diseases of abnormal bowel function utilising meal or protein derived from Asteraceae.

The present invention relates to the use of glucans for treating or preventing Inflammatory Diseases of the intestinal tract. The present invention also relates to the use of meal, particularly protein, derived from Asteraceae for treating or preventing Inflammatory Diseases of the intestinal tract.

Glucans are a heterogeneous group of glucose polymers found in the cell walls of plants, bacteria and fungi. The basic structural unit in beta-glucans as described herein is a backbone chain and side chains comprising or consisting of β(1→3)-linked glucosyl units. Depending upon the source and method of isolation, beta-glucans have various degrees of branching and of linkages in the side chains. The frequency and type of linkage in the side chains determine the molecule's biological activity. Beta-glucans of fungal and yeast origin are normally insoluble in water, but can be made soluble either by acid hydrolysis or by derivatization introducing foreign groups like -phosphate, -sulphate, -amine, -carboxymethyl and so forth to the molecule.

In Europe, Asia and USA, beta-glucans especially from Bakers' yeast have long been employed as feed additives for animals, as dietary supplement for humans, in treatment of wounds, and as an active ingredient in skin cream formulations. Further, glucans have been employed as functional pharmaceutical agents exemplified by their application for treatment of cancer as shown in WO02058711. Beta-glucans are, in this context, regarded as immunostimulants increasing the activity of white blood cells partly by inducing well regulated and local inflammatory reactions.

Ulcerative colitis and Crohn's disease are incurable chronic diseases of the intestinal tract. The two diseases are often grouped together as the two major conditions which make up inflammatory bowel disease (hereinafter designated as IBD) because of their similar symptoms. It is assumed that as many as 4 million people worldwide suffer from a form of IBD. In Norway respectively 600 and 300 people each year are diagnosed ulcerative colitis and Crohn's disease. The cause of IBD is presently not known. The present state of the art defines environmental, nutritional and genetic factors and even smoking and bacterial/viral infections as possible causes for the disease. The diseases are most common in persons of Caucasian race, in women, and in younger persons.

At present IBD is not cured, it is managed, through careful control of the patient's environment, continuous control of the bowel function and through medication. Usual medication includes anti-inflammatory drugs and immunosuppressive agents. In many cases also surgical procedures, like removing parts of the bowel, are used to fight that disease.

Recent findings suggest that IBD is caused due to the lack of traditional targets such as parasites and worms. The immune system therefore turns to other targets in the gut. This hypothesis is closely related to the hygiene hypothesis which is widely applied to conditions like asthma and allergy.

Also prebiotics and probiotics are of increasing interest in the treatment of IBD.

Another aspect of the present invention is treatment of diseases related to the digestive tract, in particular abnormal intestinal function or alterations of the digestive tract in animals, like fish and mammals. A presently dominating issue is the ever growing use of plant proteins like soy and sunflower meal as a nutritional ingredient in feeds for fish and other animals, e.g. mammals. Soy products are valuable ingredients in feeds for carnivorous, omnivorous and herbivorous fish and other animals because of their low price, high content of available protein with a well balanced amino acid profile, constant composition and steady supply.

In recent years, the concentration of plant proteins in animal feeds has increased dramatically leading to unwanted side effects like inflammations and other conditions and, as a result, a reduction of weight gain, feed conversion and efficiency, and the performance of farmed animals. Plant proteins contain substances, like a broad spectrum of antinutritive factors (ANF), which stress the fish physiology and lead to adverse reactions in the animal's digestive system.

Plant protein meals may also induce morphologic changes in the intestines of animals, as seen when feeding soy to fish. With regard to fish, this pathogenesis is classified as a non-infectious sub-acute inflammation, characterized by increased proliferation, turnover and, as such, an increased number of immature cells in the mucosa of the digestive system. This results in a reduced reabsorption of endogen compounds, e.g. digestive enzymes, in the mucosa and the intestinal surface area becomes reduced. The bacteria composition in the intestines is also changing. This condition may weaken the fish's resistance to disease and seems to involve immunological mechanisms which are like those similar to hypersensitive reactions.

For most of these plant proteins, for instance soy and sunflower meal, the usual level of plant protein addition to the feed is around 10% as a practical commercial limitation.

In addition to a higher level of plant protein in animal feed, other ingredients in animal feed may lead to detrimental effects in the animals digestive system. These are exemplified by environmental toxins or generally higher concentrations of other existing feed ingredients like carbohydrates.

Even though glucan technology had its origin in the middle of the last century, specific modes of action and effects of different glucans in different environments and their function in relation to many different diseases and conditions have not yet been completely fully investigated. The only certainty in the art is that glucans, as such cannot be regarded as one isolated group of molecules as glucans having different molecular structures and origins have very different effects in relation to a large variety of diseases and conditions.

The exact mode of action is still not known. Some theories focus on the origin of the glucans as being perceived as virulent pathogens by the immune system, others focus on the molecular level, e.g. the number, type or sterical position of the side chains. Others focus on triple helical structures of glucan units, while others again refer to characteristics like, amongst other factors, molecular weight and receptor-binding affinity. Previous studies have shown that the origin and as such the common molecular structure of different glucans might be a denominator for their function against different diseases.

Amongst this uncertainty and variation in activity, the inventors of the present invention found that IBD might be treated by using a certain class of glucans having a specific molecular structure.

The glucans of the present invention have a beta-1,3 backbone, i.e. the backbone is made up of beta-1,3 linked glucopyranose units. The glucans have one or more beta-1,3 side chains, i.e. side chains attached to the backbone via a beta-1,6 linkage and where the side chains are made up of beta-1,3 linked glucopyranose units. The side chain comprises 2 or more, typically 3, 4, 5, 10 or more beta-1,3 linked glucopyranose units.

The invention provides a glucan having a beta-1,3 backbone with one or more beta-1,3 side chains linked thereto for use in the treatment of IBD or related diseases of abnormal bowel function. Preferably IBD is treated in humans and in non-human animals bowel disease or abnormal bowel function with components in common with human IBD is treated. IBD itself has been diagnosed, and therefore may be treated, in some animals, including pets such as dogs and cats.

In the present invention the glucan is administered to a subject by any possible mode of administration, but preferably orally.

The medicament may be administered as part of a dietary regimen. The medicament may be formulated as a nutraceutical, animal feed, food, part of a nutraceutical, animal feed or food and/or adjuvant. The glucan containing medicaments may be administered to any animal, including humans, non-human primates and other mammals, domestic and livestock animals, birds, and fish, including farmed birds and companion birds like parrots, and farmed fish and pet fish. Specific examples include dog, cat, horse, cow, pig, goat, rat, mouse and sheep. The present invention clearly shows that these types of glucans can be used to prevent and/or treat IBD and related diseases in mammals and fish as exemplified further below in this specification. Mammals, in particular humans are preferred targets for treatment.

The yeast glucans used in the present invention may be in their natural state, like i.e. in whole yeast or they might be processed in the sense that either the glucans are isolated from other cell components, the glucans are derivatized and/or that the chemical structure is altered as compared to the naturally occurring structure. A derivatized glucan would preferably contain the following groups: sulfate, amine, acetate, phosphate, phosphonate or carboxylmethyl. Further alterations of the chemical structure of the glucans will typically comprise reductions in length of the backbone and/or in length or complexity of branches and/or side chains.

Preferably the glucan is not in its natural state, i.e. not present as whole cells or even a whole cell wall fraction, but processed to be partially isolated from other cell wall components which it is found with in nature, for example proteins and chitin. Acid or alkali treatment or enzymatic treatments result in preferred glucans for use according to the present invention. Molecular weights of such glucans are given below.

The glucans can be from a variety of different sources, but preferably are from yeast, as exemplified by Saccharomyces cerevisiae.

The glucans of the invention include soluble and particulate glucans, both of which are effective. Without being bound by theory, it is believed that soluble and particulate glucans use the same mechanism for their action in the treatment of inflammatory diseases of the intestinal tract.

Preferred beta-glucan containing products for use according to the invention contain at least 75%, preferably at least 80%, carbohydrate as a percentage of total cell components. Of this carbohydrate, the majority is glucan.

Examples of useful beta-glucan products for the present invention include, but are not limited to, the glucan products Imucell as manufactured by Biothera and Immiflex (formerly Fluflex) as distributed by CarePharma Co, Ltd.

Examples of useful beta-glucans include, but are not limited to, particulate and soluble yeast cell wall glucans as described in PCT/IB95/00265 and EP 0759089. Depending upon yeast strain and type, glucan constitutes up to 25% of the yeast cell wall dry weight. During the process of isolating beta-glucan from yeast the outer layer of mannoprotein is removed as well as most of the inner content of the cell, leaving a “ghost” particle, or whole glucan particle, constituting the beta-glucan layer. An example of such beta-glucans include, but is not limited to, the beta-1,3/1,6 glucan product marketed as APG 3-6 by the company Biothera. If the beta-glucan is isolated from autolysed yeast, the cell wall is more collapsed giving a crumpled ghost particle.

Other yeasts which provide a source for the glucan include Brewers yeast, Candida sp. like Candida albicans, Candida cloacae, Candida tropicalis, Candida utilis, Hansenula sp. like Hansenula wingei, Hansenula arni, Hansenula henricii and Hansenula americana, Histoplasma sp., Kloeckera sp., Kluyveromyces sp. like Kluyveromyces lactis, Kluyveromyces fragilis, Kluyveromyces polysporus, Pichia sp., Rhodotorula sp., Saccharomyces sp. like Saccharomyces delbruekii, Saccharomyces rosei, Saccharomyces microellipsodes, Saccharomyces carisbergensis or different Saccharomyces strains like Saccharomyces cerevisiae R4 (NRRL Y-15903) and R4 Ad (ATCC No. 74181), Schizophyllum sp., Schizosaccharomyces sp. like Schizosaccharomyces pombe, Torula sp. and Torulopsis sp.

Other sources of glucan are mushrooms or other fungi, algae, grasses or bacteria having the molecular structure as defined in the present invention being a beta-1,3 linked glucan backbone with one or more beta-1,3 side chains linked thereto through a beta-1,6-linkage.

In the animal feed and farming industry the cells of organisms, most often yeast cells, are used, and fed directly to the animals. These products come in different forms and shapes, like compressed, liquid, crumbled, dry, active, in-active cells, and combinations like active dry, instant active dry and inactive dry. These products are most often the remnants of the cells used for other production processes like brewing or baking and are considered valuable glucan sources. The glucans of the present invention might as well be used in a non-purified manner meaning as whole cell, production intermediate, partially treated intermediate together with other components or as extracted glucan product completely or partially separated from other cell components. Particularly for non-human uses; more processed glucans are preferred.

It is convenient to use processed products or cell extracts to achieve the effect of the present invention. These products may be hydrolysed or autolysed cells, partially or completely purified cell walls. All these products are available in various forms suited to different types of use: liquid, semi-paste, paste, fine powder, oil-coated powder, micro-granulated powder, to mention only some.

Products containing isolated carbohydrate components may be combination products of two or more components (e.g. from the yeast cell wall), for example a combination of glucan and mannan.

Mannan is a polysaccharide containing a high proportion of mannose sub-units. Preferably it is made up of D-mannose, D-glucose and D-galactose at a ratio of approximately 3:1:1.

The glucan may be mixed with other components e.g. other parts of the cell wall such as mannans or components not being part of the cell walls, like vitamins or minerals and other agents frequently used in the pharmaceutical, the nutraceutical, food, animal feed and veterinary industry. Examples of this group of products are ready to use glucan-products combined with minerals and vitamins as well as nutraceuticals combining glucans and other anti-IBD agents.

In addition to the 1,3 linked side chains, the glucans may also have one or more 1,6 linked side chains. However, preferred glucans are those which have been treated by acid or enzyme or any other suitable method to significantly reduce or eliminate the number of repetitive (1,6)-linked glucose molecules within the glucan, or occur naturally with low levels of 1,6 linkages. These (1,6)-linked glucose molecules are mainly in a beta-conformation, and would normally be found in the side chains of the beta-glucan molecule. The number of beta-1,6-linked glucose moieties can vary from one to a significant proportion of the glucose moieties depending on the source of glucan. The resulting preferred glucans have beta-1,3-main chains and beta-1,3 side chains which are linked thereto through a single beta-1,6-linkage which is not cleaved off by the elimination treatment. These products can be particulate, semi-soluble or soluble. These modified glucan molecules are preferably derived from S. cerevisiae.

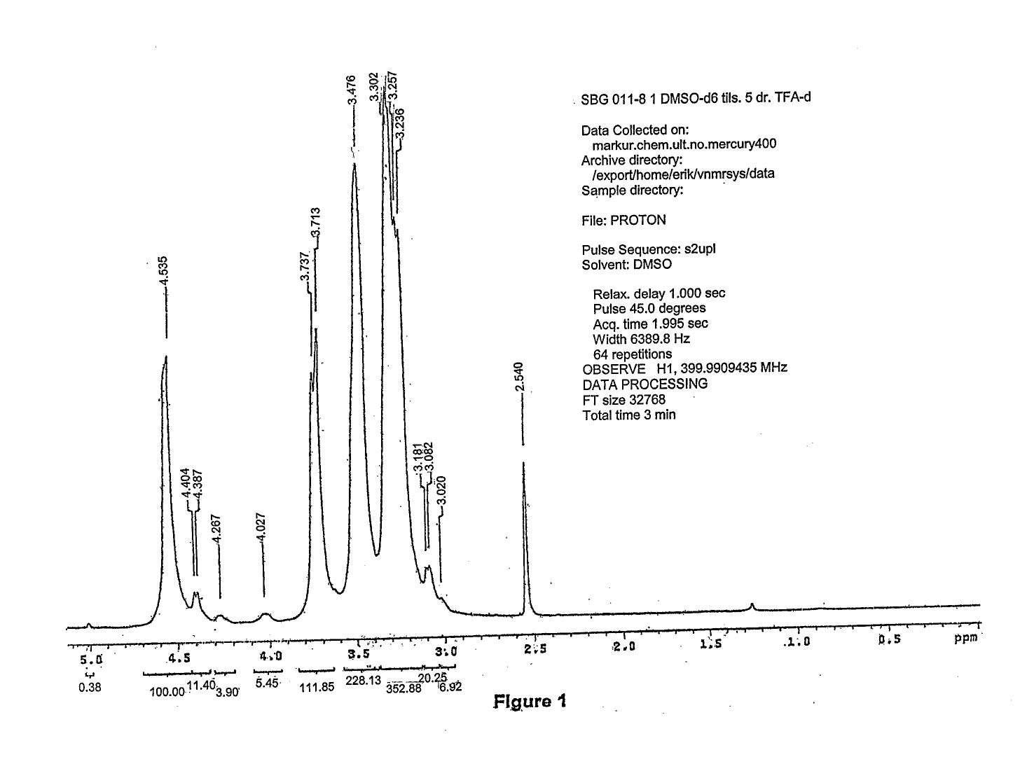

The preferred glucans are essentially free of repetitive beta 1,6-linked glucosyl units. Thus, the 1,6-linkages at the branch points do not provide ‘repetitive’ beta 1,6-linked glucosyl units but could, together with an adjacent residue, provide ‘repetitive’ beta 1,6-linked glucosyl units. By ‘essentially free’ is meant less than 2%, preferably less than 1% of the total glucosyl units. An example of such a product is seen in FIG. 1 being a ¹H-NMR-spectrum of a branched beta-1,3-glucan with <1% repetitive beta-1,6-linked glucosyl units.

Thus, preferably less than 10% more preferably less than 5%, most preferably less than 3% or 2% of the glycosidic bonds in the glucan molecule will be (1,6) linkages.

Some treatments, such as enzyme treatments, may leave up to 4 beta-1,6-linked glucosyl units uncleaved in the side chains. Such molecules are also ‘essentially free’ of repetitive beta 1,6-linked glucosyl units.

The glucan which can be used in relation to the present invention could be in the form of a single, extracted fraction or two or more different fractions with different molecular weights.

The most preferred source for the glucan for the present application are cell walls from Saccharomyces cerevisiae. Of these, a preferred source for use in the present invention is the soluble yeast product SBG (Soluble Beta Glucan) as produced by Biotec Pharmacon ASA, a Norwegian based company.

The product is an underivatized (in terms of chemical modifying groups) aqueous soluble beta-1,3/1,6-glucan, characterised by NMR and chemical analysis to consist of polymers of beta-1,3-linked D-glucose containing side-chains of beta-1,3 and beta-1,6-linked D-glucose, wherein the number of beta-1,6 moieties in the side chains (not including at the backbone/side chain branch point) is considerably reduced as compared to the structure of said glucan in the yeast cell wall. An example of such a composition is as follows:

| Component | Value/range | | typical value |
|---|---|---|---|
| WATER | 977-983 | gram/kg | 980 |
| CARBOHYDRATES | 18-22 | gram/kg | 20 |
| PROTEINS | max 1 | gram/kg | <1 |
| ASH | max 1 | gram/kg | <1 |
| LIPID | Max 1 | gram/kg | <1 |

The molecular structure of SBG is as follows:

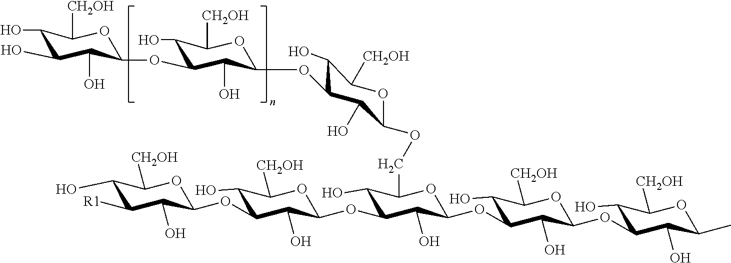

n≦0; R═H or (C₆H₈₋₁₀O₅)ₘ; m (R1+R2)=35 to 2000 glucosyl units

The reduction of the beta-(1,6)-linked glucosyl residues to produce the above preferred glucan of the present invention may be achieved in one of the following ways:

i) Enzymatic treatment, for example as described in Norwegian Patent No. 300692:
The side chains of beta-1,6-linked glucose in the micro-particulate product prepared as in U.S. Pat. No. 5,401,727 are selectively removed by enzyme treatment with an enzyme which specifically acts on beta-1,6-linkages in a poly-glucose chain. The micro-particulate product (0.2 grams) is suspended in 40 ml 50 mM ammonium acetate buffer at pH 5.0 and mixed with 20 units of the beta-1,6-glucanase enzyme. The mixture is continuously stirred for 6 hours at 37 degrees Celsius and the action of the enzyme stopped by boiling for 5 minutes. The residual enzyme treated particles are washed repeatedly in sterile distilled water by centrifugation and re-suspension. The resulting product is a branched beta-1,3-glucan with beta-1,3-glucan side chains connected by beta-1,6-linked at the branching points, and being essentially free of beta-1,6-linked glucose in the side chains which extend from the branching points. The key step being incubation of a particulate glucan with a beta-1,6-glucanase enzyme at 32 to 40° C. for 3 to 9 hours.
ii) Formic Acid treatment: For example, a micro-particulate product prepared as in U.S. Pat. No. 5,401,727 may be suspended in formic acid and heated. The suspension is cooled and free formic acid removed.

A preferred glucan containing formulation for use in the invention is a mixture of soluble beta-glucan molecules with molecular weights, (MW)>6000 daltons that interact to give a higher order conformation. For example, a mixture of linear beta-1,3-glucan chains with a numerical MW>6 kDa, preferably with a MW ranging from 6-30 kDa, together with branched high molecular weight beta-1,3-glucans (e.g. MW>15 kDa) with beta-1,3 linked side chain(s) extending from within the main chain as shown above.

Preferably, the glucans have an average molecular weight of single chains of about 20 kDa, with a range from about 6 to about 30 kDa, preferably from about 15 to about 25 kDa. In the single strain format the glucans may exist as a mixture of conformations including random coils, gel matrices or aggregates, triple helices and single helices. When in aqueous solution the molecules may take part in interchain interactions giving a high molecular weight appearance of up to 5 000 kDa when analysed by gel performance chromatography. Preferred compositions are those that form a gel like appearance in aqueous solution, demonstrating complex intermolecular interactions.

Yet another preferred product for use in connection with the present invention is NBG (Norwegian Beta Glucan), a particulate yeast product as produced by Biotec Pharmacon ASA. NBG is a product derived from Bakers Yeast (Saccharomyces cerevisiae). The product is a natural underivatized (in terms of chemical modifying groups) particulate beta-1,3/1,6-glucan, characterised by NMR and chemical analysis to consist of polymers of beta-1,3-linked D-glucose containing side-chains of beta-1,3 and beta-1,6-linked D-glucose.

Typical values for the chemical composition of NBG are as follows:

| Component | % by weight | Typical range % |
|---|---|---|
| CARBOHYDRATES | Min 75 | 75-80 |
| LIPIDS | Max 5 | 3-5 |
| NITROGEN | Max 1.4 | 0.8-1.2 |
| ASH | Max 12 | 8-10 |
| TOTAL SOLID | Min 95 | 95-98 |

The basic common molecular structure of SBG and NBG, preferred beta-glucans for use in the present invention, is as follows:

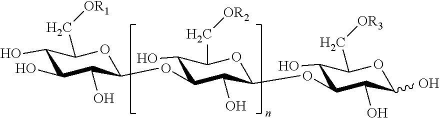

R═H or (C₆H₈₋₁₀O₅)₁₋₅₀; n=35-2000;

SBG and NBG are particularly suitable for administration to humans.

A further preferred source of glucan for use in the present invention, particularly for administration to non-human recipients, is the yeast product PatoGard™ as sold by Immunocorp, a Norwegian based company. The composition of said product is as follows:

| Component | weight % |
|---|---|
| Carbohydrate: | min 40 |
| Protein: | max 32 |
| Ash: | max 8 |
| Lipids: | max 15 |
| Moisture: | max 8 |

Typical values for the carbohydrate components are as follows:

| Component | % of total carbohydrates |
|---|---|
| Glucan | 20 |
| Mannan | 25 |
| Chitin | <1 |
| Glycogen | <2 |

Typically PatoGard™ comprises approximately 20% to 30% by weight protein, 20% to 35% beta-glucan and 20% to 35% mannose.

A further preferred source, again especially for non-human recipients, is the hydrolyzed yeast product MacroGard® Feed Ingredient as sold by Immunocorp, a Norwegian based company. The composition of said product is as follows:

| Component | % by weight | typical range % |
|---|---|---|
| CARBOHYDRATES | min 60 | 63-68 |
| BETA-1,3/1,6- | min 54 | 57-61 |
| GLUCAN | | |
| LIPIDS | Max 18 | 13-18 |
| PROTEIN | Max 8 | 5-7 |
| ASH | Max 12 | 6-10 |
| TOTAL SOLID | min 92 | 94-97 |

An alternative is MacroGard®Pet which has the following composition:

| Component | % by weight | Typical range % |
|---|---|---|
| Carbohydrates | min 65 | 65-70 |
| Lipids | max 15 | 12-14 |
| Protein | max 8 | 5-7 |
| Ash | max 10 | 5-9 |
| Dry matter | min 92 | 94-97 |

A further preferred source of glucan is MacroGard®AquaSol, which has the following composition:

| Component | % of dry matter | Typical range % |
|---|---|---|
| β-1,3/1,6-Glucan | min 95 | 96-99 |
| Lipids | max 1 | 0-1.0 |
| Ash | max 2 | 0.1-1.0 |
| Protein | max 1 | 0-1.0 |

Other MacroGard® products include MacroGard®Immersion Grade, MacroGard® Adjuvant, and MacroGard® F1 Suspension. MacroGard® Feed Ingredient is particularly preferred.

PatoGard® and MacroGard® are both suitable for all the methods and uses described herein.

In some instances, yeast glucan products in which the glucan is substantially purified, such as MacroGard™ as sold by Immunocorp, and similar products, are preferred. Such products can be defined in terms of the ratio of their total yeast cell wall-derived glucan and mannan content, i.e. essentially their total carbohydrate content to their total yeast cell wall-derived protein content. Typically, such products have a ratio of total yeast cell wall-derived carbohydrate content to total yeast cell wall-derived protein content of at least 7:1, preferably at least 10:1 or 12:1, e.g. around 15:1.

For other applications, yeast glucan products in which the glucan is purified to a lesser extent, such as PatoGard™ as sold by Immunocorp, and similar products, are preferred. Typically, such products have a ratio of total yeast cell wall-derived carbohydrate content to total yeast cell wall-derived protein content in the range of approximately 1:1 to 7:1, preferably 1:1 to 5:1.

The preferred particulate beta-glucan of the present invention may be prepared in the following way:

By repeated extractions in alkali and acid of dry Saccharomyces cerevisiae, for example according to the procedure described in U.S. Pat. No. 5,401,727 (incorporated herein by reference). The extraction process described removes cytoplasmic components inside the yeast cells as well as the mannose containing polysaccharides and proteoglycans which are on the cell surface. The product prepared according to this procedure consists of a beta-1,3 beta-1,6-glucan with a particle size of 2-5 micrometers. The chemical structure of this micro-particulate beta-1,3 beta-1,6-glucan is characterized by 83% beta-1,3 linked glucose, 6% beta-1,6 linked and 5% beta-1,3,6 linked'glucose, and it is a beta-1,3-glucan chain with beta-1,3,6-linked glucose as the branch points.

The particulate glucans of the present invention have a molecular weight in the range of 5000 Da to 1,000,000 Da, preferably in the range of 25 kDa to 500 kDa, more preferably in the range of 150 kDa to 300 kDa and most preferably about 250 kDa.

The particulate glucans described above may be solubilized as described in WO/2001/062283 (incorporated herein by reference). Thus, formic acid can be used to both reduce the number of beta-(1,6)-linked glucosyl residues in the glucan and to solubilize the glucan.

Other structures and/or structural conformations within the family of beta-1,3-glucans can be readily identified or isolated by a person of ordinary skill in the art following the teaching of this invention. The above is thus a guideline to achieve a highly potent product, but is not a limitation towards even more potent products. Isolated structural elements of the complex mixture may have improved effects over the presently exemplified formulations when administered.

Suitable carriers or auxiliaries for use in formulating glucan containing compositions for use in the present invention include magnesium carbonate, titanium dioxide, lactose, mannitol and other sugars, talc, milk protein, gelatine, starch, vitamins, cellulose and its derivatives, animal and vegetable oils, polyethylene, glycols and solvents, such as sterile water, alcohols, glycerol and polyhydric alcohols. The pH and exact concentration of the various components of the composition are adjusted according to routine skills.

The compositions for medical and veterinary use are preferably prepared and administered in dose units. The term “dose units” and its grammatical equivalents as used herein refer to physically discrete units suitable as unitary dosages for the human or non-human subject, each unit containing a predetermined effective amount of glucan calculated to produce the desired therapeutic effect in association with the required physiologically tolerable carrier, e.g., a diluent or a vehicle.

The composition may comprise the active ingredient alone, in a form suitable for administration to a subject, or the composition will typically comprise the glucan and one or more physiologically acceptable carriers, one or more additional active ingredients, or some combination of these.

The formulations described herein may be prepared by any method known or hereafter developed in the art of pharmacology, veterinary science, animal and human nutrition etc. In general, such preparatory methods include the step of bringing the active ingredient into association with a carrier or one or more other accessory ingredients, and then, if necessary or desirable, shaping or packaging the product into a desired single or multi-dose unit. Controlled or sustained-release formulations of a composition of the present invention may be made using conventional technology.

Dosage levels of the active compounds comprised in the composition for use in the present invention may vary. Functional dose ranges of the glucans can be readily determined by one of ordinary skill in the art. For example, when administered orally the functional dose range and effective amount for a human would be in the region of 0.1-500 mg/kg b.w. (body weight)/day, preferably 1-100 mg/kg b.w./day, most preferably 5-30 mg/kg b.w./day. When administered parenterally a suitable functional dose range would be 0.1-10 mg/kg b.w./day.

The compositions according to the invention may be presented in the form of an article or carrier such as a tablet, coated tablet, lozenges, troches, syrups or elixirs, liposomes, powder/talc or other solid, solution, emulsions, suspension, liquid, spray, gel, drops, aerosol, douche, ointment, foam, cream, gel, paste, microcapsules, controlled release formulation, sustained release formulation or any other article or carrier which may possible or useful in light of the, at any give point in time and intended, preferred mode of administration.

The route(s) of administration will be readily apparent to the skilled artisan and will depend upon any number of factors including the type and severity of the disease being treated, the type and age of the subject being treated, and the like. The most preferred route of administration is orally, optionally by gavage.

Formulations suitable for oral administration of the glucan (preferably soluble glucan) include, but are not limited to, an aqueous or oily suspension, an aqueous or oily solution, or an emulsion. Such formulations can be administered by any means including, but not limited to, soft gelatin capsules. Liquid formulations of a pharmaceutical composition of the present invention which are suitable for oral administration may be prepared, packaged, and sold either in liquid form or in the form of a dry product intended for reconstitution with water or other suitable vehicle prior to use. With regard to the particulate product, it is possible to use other means of administration including but not limited to capsules, tablets, powders, granules, lozenges, drops, suppositories or any other means of administration suitable for a particulate product.

Therapy may be repeated intermittently while the symptoms are present or even when they are not present. It might be relevant to administer the components two weeks prior to the expected challenge and/or for several weeks after the challenge. Continuous use is also possible, as for the treatment of chronic conditions.

The glucan may be provided alone or in combination with other medicaments to provide an operative combination. Thus in a further embodiment is provided a product containing (a) a glucan as described above, and (b) a second active agent for the treatment of IBD or related diseases of abnormal bowel function, as a combined preparation for simultaneous, separate or sequential use in the treatment of IBD or related diseases of abnormal bowel function. Preferably the second active agent is derived from the plant family Asteraceae and, preferably is a protein containing fraction therefrom, i.e. a meal, e.g. sunflower meal, a product from which the oil has been largely removed.

Thus, it is possible to use a single glucan, a combination of two or more glucans or, if applicable, a combination of glucan(s) and another medical substance. With regard to a composition including two or more glucans it is possible to use different glucans from the same or different species or from the same species but produced by different methods.

A skilled artisan/physician will be able to select the medical substances which can be applied together with glucans for treatment of the relevant condition. Examples of suitable additional medical substances are, but are not limited to, immunosuppressive agents like azathioprine (Imuran), methotrexate (Folex, Rheumatrex), or 6-mercaptopurine (Purinethol, 6-MP) and cyclosporine A (Sandimmune, Neoral); sulfasalzine (Azulfadine), mesalamine (Asacol, Pentasa) and Olsazine (Dipentum); steroids like corticosteroids exemplified by prednisone, methylprednisolone or budesonide (Entocort EC), antibiotics like metronidazole or tylosin; biologics like the intravenously administered infliximab (Remicade); as well as alternative and different medication used in complementary medicine like fish oil and other agents including, but not limited to, aloe vera, butyrate, boswellia, probiotics and nicotine.

In general, the beta-glucan can be administered to an animal as frequently as several times daily, or it may be administered less frequently, such as once a day. The treatment will for instance depend upon the type of IBD or related disease, the severity of the condition, and the condition of each patient. The glucan treatment may be closely interrelated with any other treatment regimen, and could be ahead of, concurrent with, or after the administration of any other medicament.

The glucan or compositions of two or more glucans as described in the present invention may be applied as prophylaxis for prevention of IBD conditions in advance of the outbreak of the disease or as a treatment after IBD has been diagnosed. Thus, ‘treatment’ or ‘treating’ as used herein includes, but is not limited to, prophylactic treatment, i.e. prevention, and also stabilisation e.g. treatment of a disease which would worsen if left untreated but which does not result in cure of the disease. ‘Treatment’ includes a measurable and beneficial improvement in one or more, preferably more than one symptom of or risk factor for IBD or a related disease of abnormal bowel function. Preferably in one or more symptoms and more preferably a conclusion by the patient and/or treating physician that the IBD or related disease is improved, either in terms of the historical presentation of the disease or what was anticipated (e.g. in the case of a prophylactic treatment).

The term “IBD” refers to Inflammatory Bowel Diseases (hereinafter designated as IBD) which are mainly comprised of two chronic diseases that cause inflammation of the intestines: ulcerative colitis and Crohn's disease. Although the diseases have some features in common, there are some important differences mainly with regard to the nature and location of the inflammatory changes. Ulcerative colitis is an inflammatory disease which is mainly restricted to the large intestine, also called the colon. In ulcerative colitis, the inner lining—or mucosa—of the intestine becomes inflamed and develops ulcers. Crohn's disease differs from ulcerative colitis in the areas of the bowel it involves—it most commonly affects the last part of the small intestine, the terminal ileum, and parts of the large intestine. However, Crohn'S disease can also attack any part of the gastrointestinal tract. Crohn's disease generally tends to involve the entire bowel wall, whereas ulcerative colitis affects only the lining of the bowel. Accounting for far fewer cases are other forms of IBD like Collagenous colitis, Lymphocytic colitis, Ischaemic colitis, Diversion colitis, Behcet's syndrome, Infective colitis and Indeterminate colitis.

‘Related diseases of abnormal bowel function’ include those diseases and conditions where patients exhibit continuous or sporadic impaired bowel function, generally associated with altered intestinal motility and/or bowel inflammation. Such related diseases and conditions include constipation, diarrhoea and fecal incontinence and conditions and diseases resulting due to a surgical bowel resection or the like.

This invention also provides a kit or an administration device comprising a glucan as described herein and information material which describes administering the glucan to a human or other animal for treatment of IBD or related diseases of abnormal bowel function. The kit or administration device may have a compartment containing the glucan. As used herein, the “Information material” includes, but is not limited to, a publication, a recording, a diagram, or any other medium of expression which can be used to communicate the usefulness of the composition of the invention for its designated use.

The Asteraceae protein described herein is itself (independently of the presence of a glucan) of utility in the treatment of diarrhoea. Such a treatment includes a relative reduction in diarrhoea as compared to that seen with comparable feeds which are not in accordance with the invention. Diarrhoea can be assessed based on the amount of dry matter in faeces. Treatment also includes prevention, the feeds preventing an otherwise expected level of diarrhoea.

The Asteraceae protein described herein is also of utility in the treatment of bowel disease and in improving bowel health. Relevant bowel diseases will typically be inflammatory and include Inflammatory Bowel Disease or Inflammatory Bowel Syndrome.

The Asteraceae is preferably from the genus Helianthus, most preferably it is Helianthus annus (sunflower).

Where the Asteraceae protein is administered as part of an animal feed formulation, Asteraceae meal will typically comprise 2-50%, preferably 5-40%, more preferably 8-30% of the total feed formulation.

Thus, in a further aspect, the present invention provides Asteraceae protein for use in treating or preventing bowel disease in an animal.

Where reference is made herein to Asteraceae, or other plant proteins, unless otherwise clear from the context, it should be understood that Asteraceae, or other plant meal may be used. In a further aspect, other components of the Asteraceae meal than the protein part may be used in place of the Asteraceae protein in various formulations and methods described herein.

The term ‘meal’ is a well known term in the art used to refer to the residue left after some or most of the oil from a plant, seed or bean etc. has been removed, e.g. in a crushing and solvent-extraction method. Thus, these plant protein sources, also commonly defined as oilseed proteins can be fed whole, but they are more commonly fed as a by-product after oils have been removed.

Sunflower meal includes protein, fibre, ash and fat and oil residues. The composition of a sunflower meal depend on the oil content of the seed, the extent of hull removal, the efficiency of oil extraction and the temperature at which the oil is removed, amongst other variables.

Thus, in a further aspect, the present invention provides Asteraceae meal or any factor derived therefrom, e.g. protein, for use in the treatment of IBD or related diseases of abnormal bowel function. Discussions above about formulation of glucans and target species for therapy apply mutatis mutandis to the use of Asteraceae derived products. Humans, fish and livestock mammals are preferred.

By “an effective amount” is meant an amount of a compound effective to ameliorate the symptoms of, or ameliorate, treat, prevent, delay the onset of or inhibit the progression of a disease. Ultimately, the attending physician or veterinarian will decide the appropriate amount and dosage regimen. The “effective amount” of the active ingredients that may be combined with the carrier materials to produce a single dosage will vary depending upon the subject treated and the particular mode of administration.

Various documents including, for example, publications and patents, are recited throughout this disclosure. All such documents are, in relevant part, hereby incorporated by reference. The citation of any given document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

The compositions described herein may comprise, consist essentially of, or consist of any of the elements as described herein.

For the purpose of this specification it will be clearly understood that the word “comprising” means “including but not limited to”, and that the word “comprises” have a corresponding meaning. Therefore the words “comprise”, “comprises”, and “comprising” are to be interpreted inclusively rather than exclusively.

As used herein and in the appended claims, the singular forms “a,” “an,” and “the” include plural reference unless the context clearly dictates otherwise.

Unless defined otherwise, all technical and scientific terms and any acronyms used herein have the same meanings as commonly understood by one of ordinary skill in the art in the field of the invention.

The following examples are intended to be illustrative of the present invention and to teach one of ordinary skill in the art to make and use the invention. These examples are not intended to limit the invention in any way. The invention will now be further described in the following Examples and the figures in which:

FIG. 1 is an ¹H-NMR-spectrum of a branched beta-1,3-glucan with <1% repetitive beta-1,6-linked glucosyl units. The different observed chemical shifts are represented in Table 1 below:

TABLE 1

| Chemical | | |
|---|---|---|
| shift (ppm) | Assignment | Comment |
| 5.00 | H1 RT(α) | H1 in the α-anomer for the |
| | | reducing terminus (RT) |
| 4.54 | H1 BC | H1 in backbone chain of (1- |
| | | 3)-linked glucosyl repeat |
| | | units (GRUs) |
| 4.39 | H1 NRT + H1 | H1 in the non-reducing |
| | RT (β) | terminus (NRT) + in the β- |
| | | anomer of the reducing |
| | | terminus (RT) |
| 4.27 | H1 (1-6) SC | H1 in (1-6)-linked side- |
| | | chains |
| 4.03 | H6 (1-6) SC | H6 in (1-6)-linked side- |
| | | chains |
| 3.72 | H6 BC | H6 in the backbone chain of |
| | | (1-3)-linked glucosyl repeat |
| | | units (GRUs) |
| 3.48 | H3 BC + H6′ BC | H3 and H6′ in the backbone |
| | | chain of (1-3)-linked |
| | | glucosyl repeat units (GRUs) |
| 3.30-3.24 | H2 BC + H4 BC + | H2, H4 and H5 in the backbone |
| | H5 BC | chain of (1-3)-linked |
| | | glucosyl repeat units (GRUs) |
| 3.09 | H2 NRT | H2 in the non-reducing |
| | | terminus (NRT) |
| 3.02 | H2 (1-6) SC | H2 in (1-6) linked side- |
| | | chains |
| 2.54 | DMSO | The solvent peak |

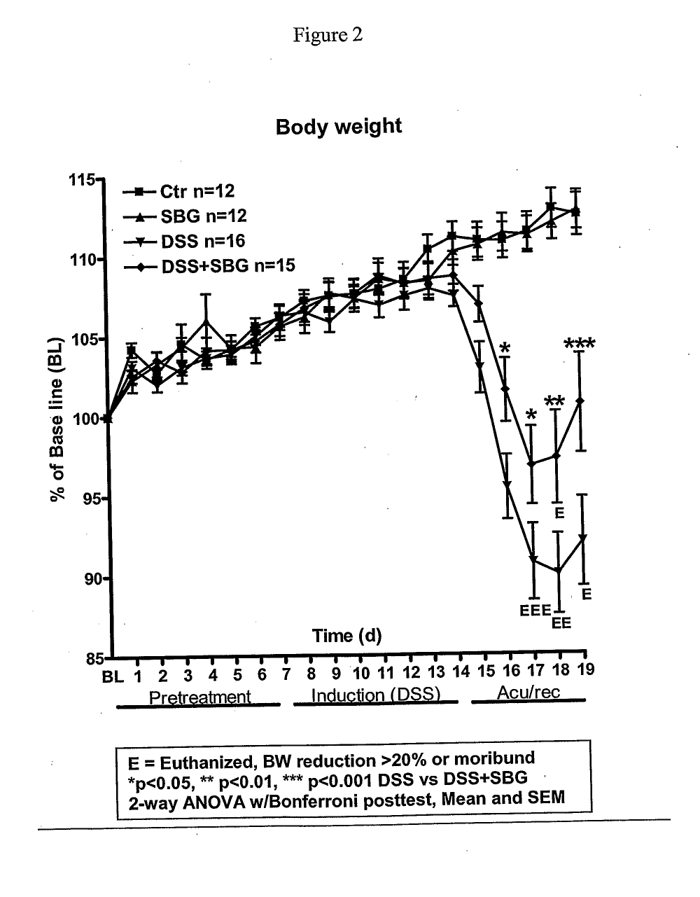

FIG. 2 shows a comparison of body weight changes in animals treated as defined in example 1 below.

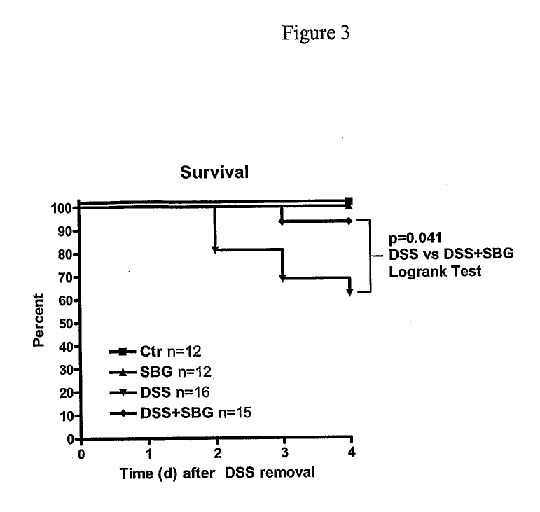

FIG. 3 shows the survival rate of animals treated as defined in example 1 below.

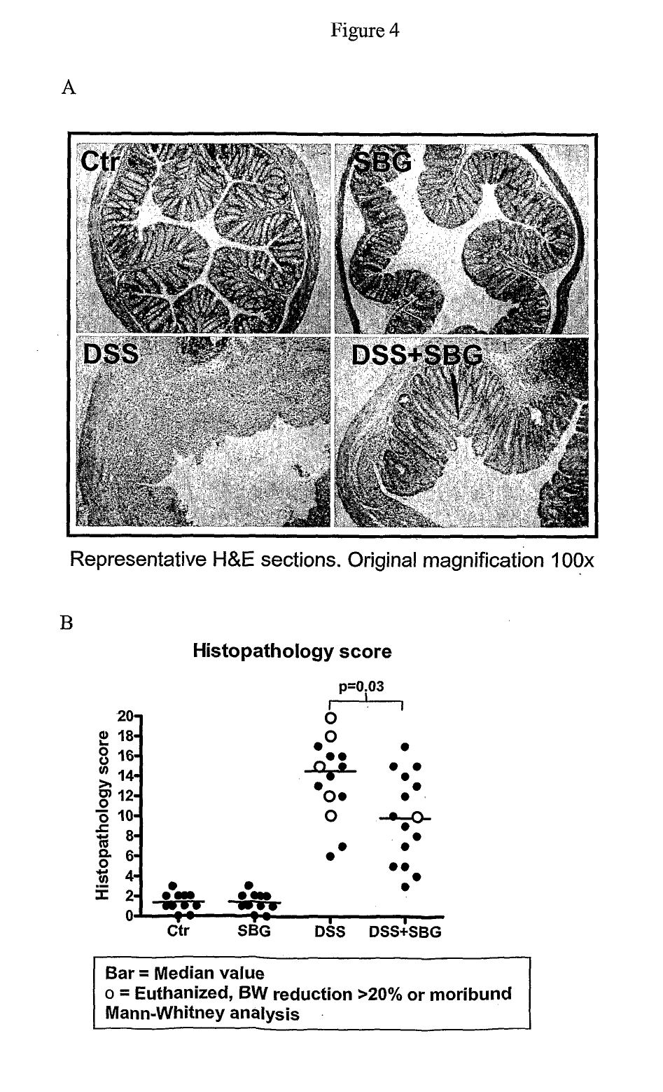

FIG. 4 shows (A) representative colon sections and (B) a graded comparison of colonic inflammation and tissue damage in acute colitis in accordance with the experimental design as defined in example 1 below.

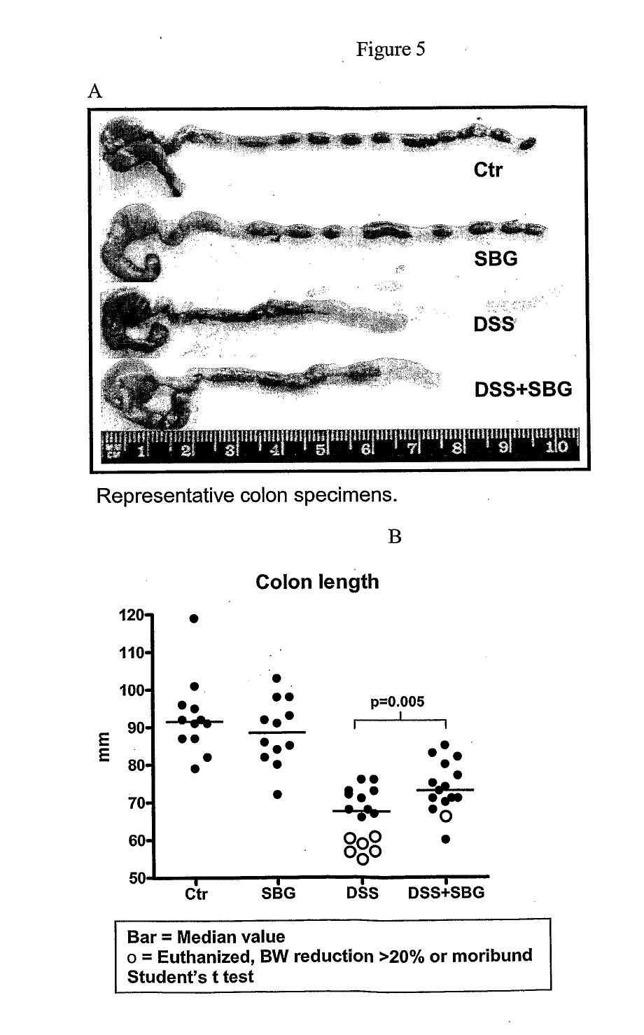

FIG. 5 shows (A) a representative colon length illustration and (B) the distribution of colon lengths of mice treated according to the model as defined in example 1 below.

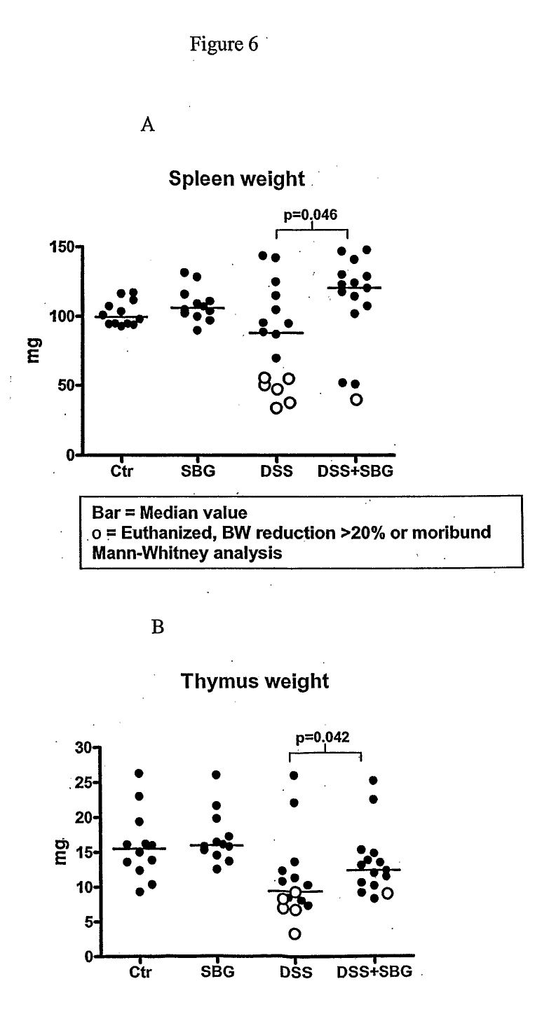

FIG. 6 shows (A) the distribution of the spleen weight and (B) thymus weight of mice treated according to the model as defined in example 1 below.

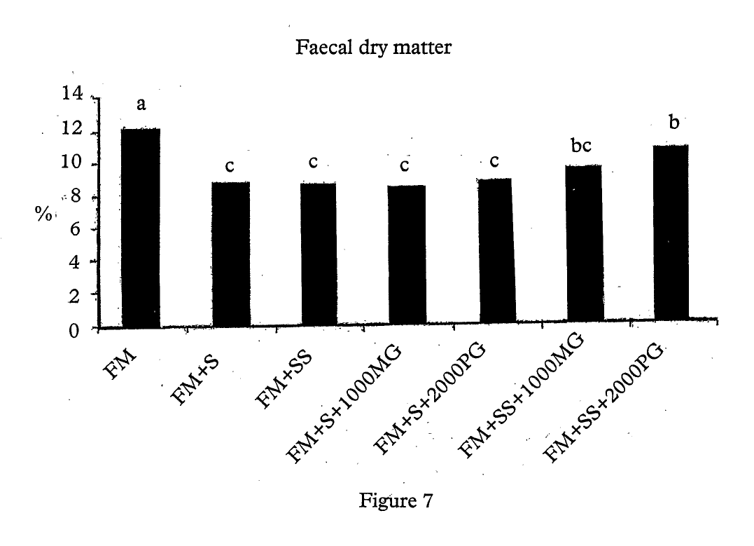

FIG. 7 is a graph showing the percentage of dry matter in the faeces of Atlantic salmon, this being a good indicator of diarrhoea.

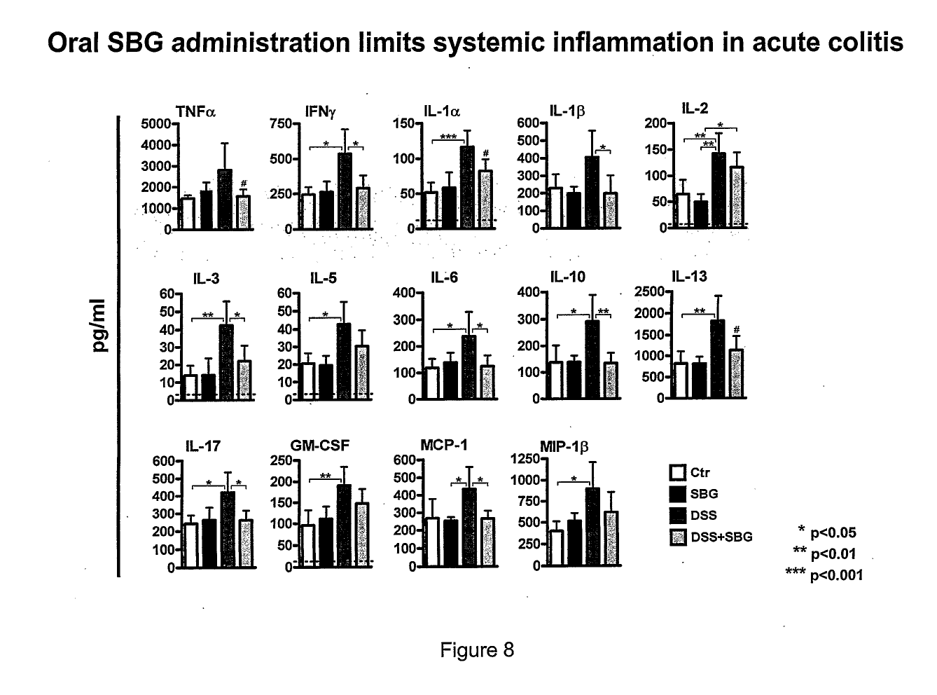

FIG. 8 is a series of graphs showing the levels of selected mediators associated with systemic inflammation in acute colitis in animals treated as defined in example 1 below.

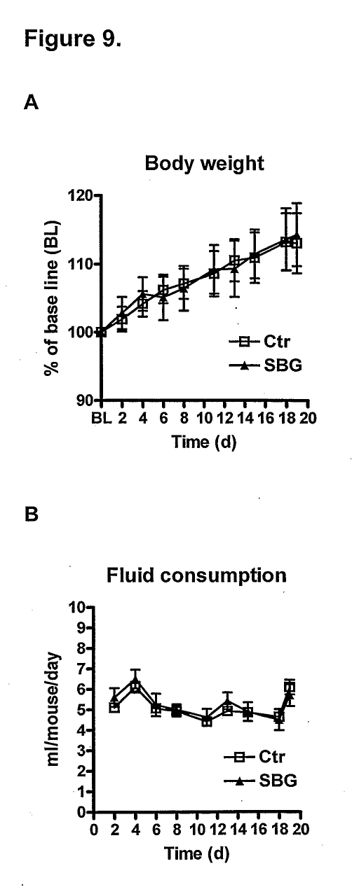

FIG. 9 shows (A) the body weight and (B) the average fluid consumption of mice treated as defined in example 4 below.

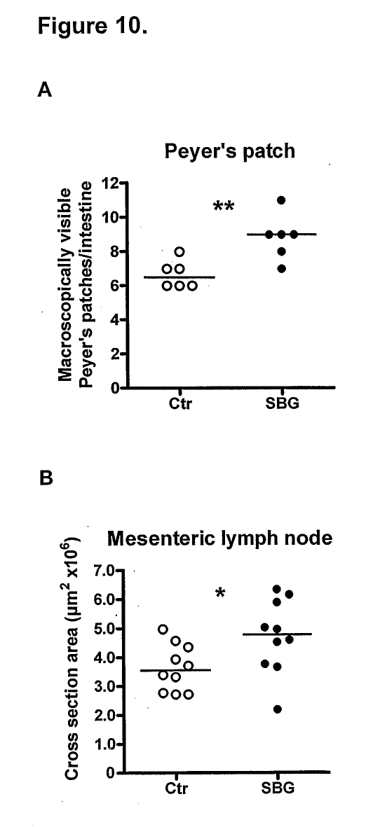

FIG. 10 shows (A) the number of macroscopically visible Peyer's patches and (B) the cross section area of formalin fixed mesenteric lymph nodes of mice treated as defined in example 4 below.

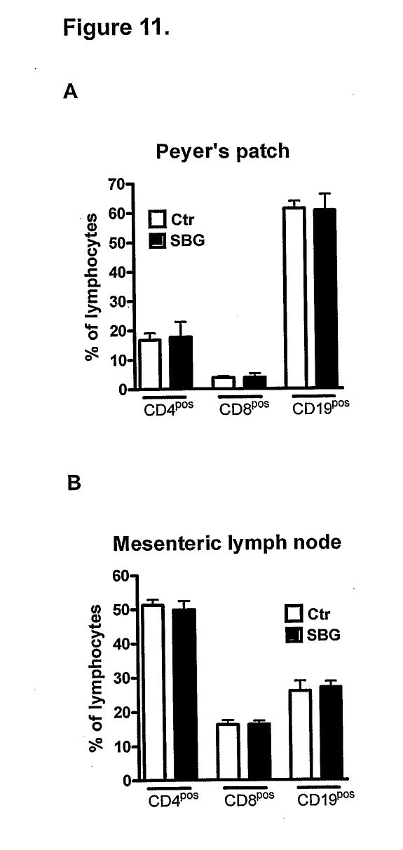

FIG. 11 shows the composition of major lymphocyte subsets of (A) the Peyer's patches and (B) the mesenteric lymph nodes of mice treated as defined in example 4 below.

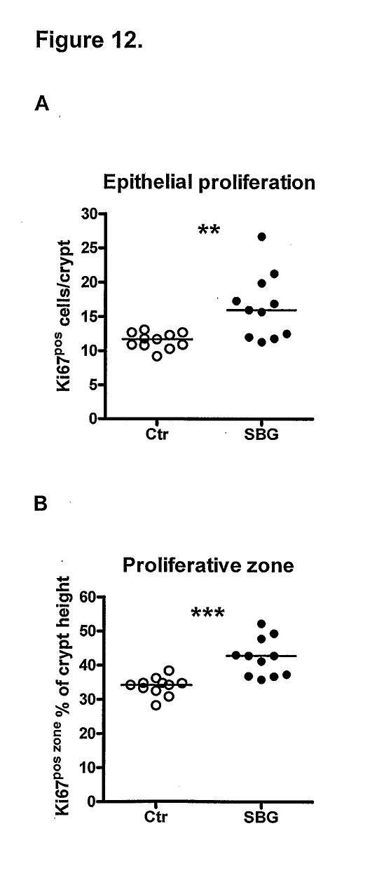

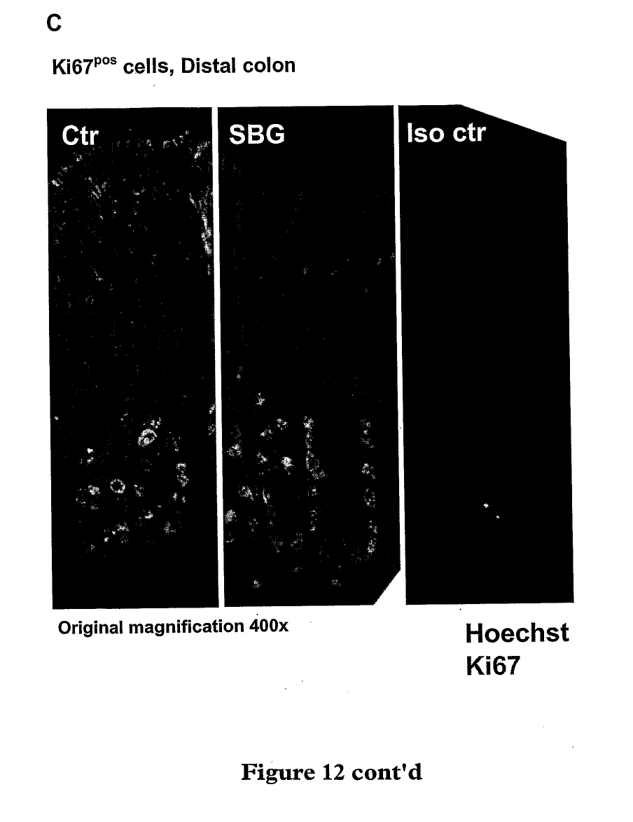

FIG. 12 shows the (A) number and (B) distribution of Ki67 positive cells, proliferating intestinal epithelial cells, in the distal colon of mice treated as defined in example 4 below; (C) images of representative stainings.

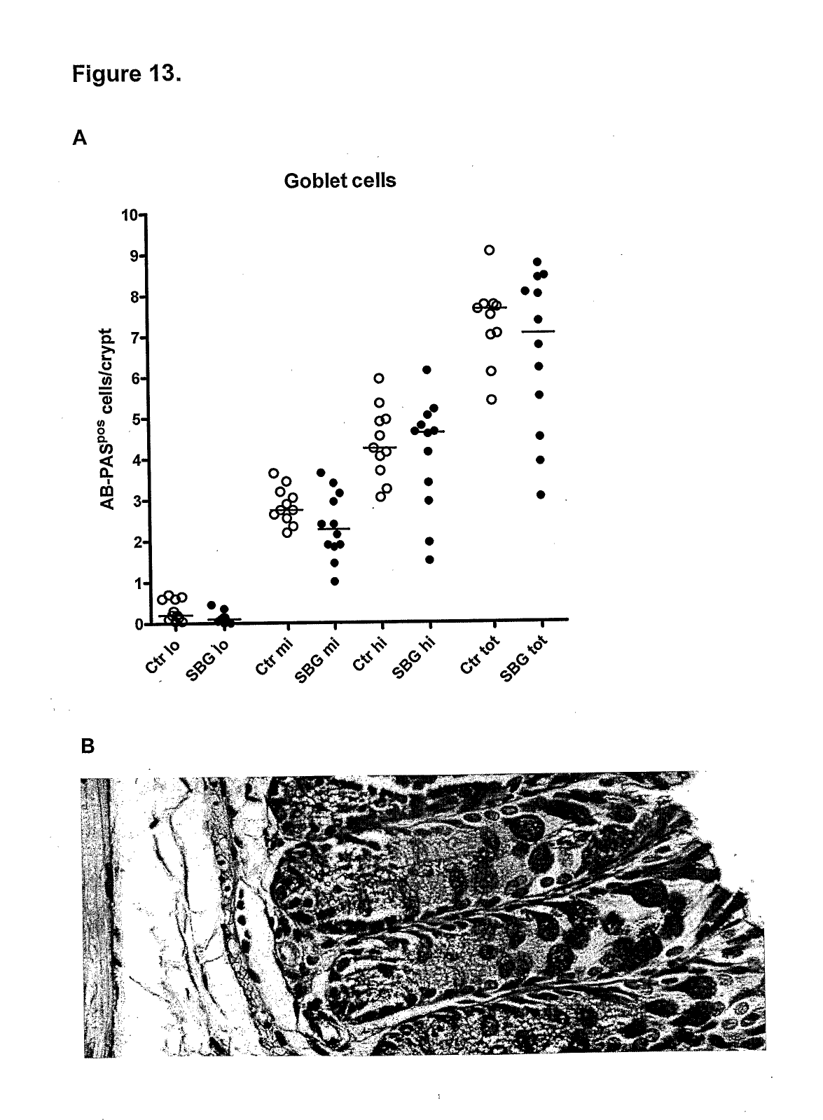

FIG. 13 shows the (A) number and distribution of AB/PAS positive goblet cells in mice treated as defined in example 4 below and (B) an image of representative staining.

### EXAMPLES

### Example 1

### Effect of Soluble Beta-Glucan on Experimental Colitis

### 1.1 Experimental Design:

A model was established to evaluate the effects of soluble glucans (here the product SBG from the company Biotec Pharmacon ASA) on treatment of inflammatory bowel disease, here exemplified by ulcerative colitis.

Experimental colitis was induced by exposure to dextran sulphate sodium (DSS) dissolved in the drinking water for 7 days. Animals were pretreated as indicated below for 7 days before induction of colitis. Mice were sacrificed following an acute-/recovery phase of 4 days.

Animals were divided into four experimental groups:
1. Control animals (n=12) were provided with regular drinking water throughout the experiment.
2. SBG treated animals (n=12) were provided with SBG (100 mg/L) in the drinking water throughout the experiment.
3. DSS treated animals (n=16) were provided DSS (1.5% w/v) during the induction period (7 d). Regular drinking water was administered during the pretreatment phase (7 d) and the acute-/recovery phase (4 d).
4. SBG+DSS treated animals (n=15) were provided DSS (1.5% w/v) dissolved in water containing SBG (100 mg/L) during the induction period (7 d). SBG (100 mg/L) in the drinking water was administered during the pretreatment phase (7 d) and the acute-/recovery phase (4 d).

### 1.2 Experimental Details:

Animals were monitored daily for general signs of morbidity and body weight and fluid consumption was recorded. Main criterion for humane endpoint was body weight reduction of >20% of baseline weight. Mice assessed as clearly moribund, without meeting the weight criterion; were also euthanized for animal welfare reasons.

Animals were anesthetized by subcutaneous injection of Hypnorm® and Midazolam (50-75 μL/10g body weight) prior to cardiac puncture. Postmortem, colon, spleen and thymus were excised. The colon was flushed with cold PBS and partitioned into proximal-, medial- and distal colon segments prior to fixation. All tissue samples were kept on ice and fixed in formalin for subsequent preparation and analysis.

The outcome was assessed by studying body weight changes, survival rates, colonic inflammation and tissue damage, disease associated colon shortening and changes in spleen and thymus weight and changes in levels of inflammatory mediators in circulation.

Colonic inflammation and tissue damage was evaluated by a trained pathologist blinded to the sample identity and study groups. The histopathological score was expressed as a combination of inflammatory cell infiltration (score 0-3), tissue damage (score 0-3), absence or presence of lymphoid aggregates (score 0 or 1) and absence or presence of epithelial regeneration (score 0 or 1). The tissue damage score was adjusted by multiplying the score with the proportion of area ulcerated (0-25%=x1, 25-50%=x2, 50-75%=x3 and 75-100%=x4) (Table 2). A total score was calculated by adding together the scores obtained from the proximal, medial and distal colon segments.

TABLE 2
Histopathology scoring criteria

| Inflammatory cell infiltration | Score 0-3 |
|---|---|
| Presence of occasional inflammatory cells in the | 0 |
| lamina propria | |
| Increased numbers of inflammatory cells in the | 1 |
| lamina propria | |
| Confluence of inflammatory cells extending into | 2 |
| the submucosa | |
| Transmural extension of inflammatory infiltrate | 3 |
| Tissue damage | Score 0-3 |
| No mucosal damage | 0 |
| Lymphoepithelial lesions | 1 |
| Surface mucosal erosion or focal ulceration | 2 |
| Extensive mucosal damage and extension into | 3 |
| deeper structures of the bowel wall | |
| Ulcerated area of epithelial surface | Factor 1-4 |
| 0-25% | 1 |
| 25-50% | 2 |
| 50-75% | 3 |
| 75-100% | 4 |
| Lymphoid aggregates | Score 0-1 |
| Absent | 0 |
| Present | 1 |
| Epithelial regeneration | Score 0-1 |
| Absent | 0 |
| Present | 1 |

Note to table 2:
A histological score, to quantify the degree of colonic inflammation and injury, was established in biopsies, originating from the proximal, medial and distal parts of the colon and combined to a total score. Where a tissue damage score of ≧2 were recorded, the score was multiplied by a factor corresponding to the area affected.

### 1.3 Results:

### Oral SBG Administration Reduces Colitis-Associated Body Weight Loss.

Body weight loss, a critical clinical symptom, was monitored to evaluate the protective effect of SBG on experimental IBD. Male BALB/c mice were pretreated with SBG or regular drinking water for 7 days, prior to induction of acute colitis by administering DSS for 7 days. Control (Ctr) animals [base line weight 22.7 (21.4-24.0) g; mean and (range), n=12], were provided with regular drinking water throughout the experiment.

SBG-treated animals [base line weight 22.2 (20.3-24.9) g; mean and (range), n=12], were provided with SBG (100 mg/L) in the drinking water throughout the experiment. DSS-treated animals [base line weight 22.3 (20.6-23.4) g; mean and (range), n=16], were provided DSS (1.5% w/v) during the induction period. Regular drinking water was administered during the pretreatment phase and the acute-/recovery phase.

DSS+SBG-treated animals [base line weight 22.1 (19.4-24.0) g; mean and (range), n=15], were provided DSS (1.5% w/v) dissolved in water containing SBG (100 mg/L) during the induction period. SBG (100 mg/L) in the drinking water was administered during the pretreatment phase and the acute-/recovery phase. Body weight and fluid consumption was recorded daily during pretreatment, colitis induction and for 4 days after DSS termination.

We observed a dramatic weight loss of approximately 15% between day 14 and 17 in DSS-treated animals. Although, animals treated with SBG prior to, during and following colitis induction experienced a moderate weight loss, the DSS+SBG group was relatively protected from colitis-associated weight loss compared to the DSS-only group (P<0.05 at day 16 and 17, P<0.01 at day 18 and P<0.001 at day 19, DSS+SBG vs. DSS) (FIG. 2).

Furthermore, the onset of weight reduction was slightly delayed in the SBG+DSS group compared to the DSS group. Non-colitis control animals increased steadily in weight throughout the experiment. We did not observe any difference in body weight dynamics between the SBG animals and mice receiving regular drinking water (FIG. 2).

To estimate the daily SBG dose and DSS exposure, fluid consumption was monitored. An approximate average of 5-7 mL/mouse/day, corresponding to a daily SBG dose of 20-30 mg/kg was recorded (data not shown).

### Oral SBG Administration Reduces Colitis-Associated Mortality.

DSS exposure induced clinical symptoms including bloody stool, diarrhea, rectal bleeding, inactivity, failure to groom and in some severe instances hunched posture and trembling. Obviously moribund animals were euthanized for animal well fare grounds. Severe, colitis-associated, body weight loss is associated with mortality, thus mice experiencing weight loss exceeding 20% of base line weight were sacrificed for humane reasons.

Oral SBG treatment increased the proportion of mice surviving to the planned endpoint, 4 days after DSS termination (P=0.041, DSS+SBG vs. DSS) (FIG. 3). In the DSS+SBG group only 1 out of 15 animals had to be sacrificed prior to the planned end point, compared to 6 out of 16 mice in the DSS group. Also, the need to sacrifice animals arose earlier in the DSS group compared to the DSS+SBG group (FIG. 3).

### Oral SBG Administration Reduces Dss-Induced Colonic Inflammation and Tissue Damage.

To investigate whether SBG administration could protect against DSS-induced inflammation and ulceration, histology sections of the proximal, medial and distal colon were examined. The presence and degree of inflammatory cell infiltration and tissue damage as well as the presence or absence of lymphoid aggregates and signs of epithelial regeneration was addressed and a histopathology score was established (see table 2).

No apparent pathology was observed in the control- or SBG groups. In the DSS group, on the other hand, considerable inflammatory cell infiltration extending into the submucosa and in several cases with transmural involvement was observed. Severe distortion of the mucosal microarchitecture, including lack of distinct crypts and goblet cells, moderate to extensive ulceration, in some cases with total lack of epithelium, was disclosed. Mucosal edema and signs of bowel wall thickening was also identified (FIG. 4 a).

Although clear histological signs of colitis was apparent in sections from DSS+SBG-treated mice, a significantly lower histopathology score was obtained in this group (P=0.027, DSS+SBG vs. DSS) (FIG. 4 band Table 3). The inflammation and tissue damage appeared more prominent in the distal part of the colon (data not shown).

### Oral SBG Administration Reduces Colitis-Associated Colon Shortening.

Colon shortening is a well established disease associated characteristic of DSS-induced colitis. To further evaluate the protective capacity of SBG on experimental colitis, colons were excised and the length was measured.

Oral SBG administration does not appear to have an effect on colon length under non-inflammatory conditions. In the DSS group colons were approximately 30% shorter than colons from control animals. Although the colon length in the DSS+SBG group was clearly affected by exposure to DSS, the colon was significantly longer than in the DSS group (P=0.005, DSS+SBG vs. DSS) (FIG. 5 a, b and Table 3).

Colon shortening correlates well with the severity of disease, as the 6 shortest colons in the DSS group all originated from animals euthanized prematurely due to disease progression. We also observed that colons from the DSS group contained largely unformed stool as opposed to fecal pellets in the DSS+SBG group and control groups.

Macroscopic wall thickening distally and loss of bowel transparency was apparent in both DSS- and DSS+SBG groups although it appeared more striking in the DSS group.

Oral SBG Administration Modulates Spleen and Thymus Weight in Acute Colitis.

To investigate whether oral SBG administration could have an impact on lymphoid tissue in acute colitis, spleen and thymus was collected postmortem. Mice euthanized prematurely due to the severity of colitis had reduced spleen- and thymus mass compared, to control animals, indicating that reduced weight correlated with colitis-associated morbidity (FIG. 6, open symbols).

No overall difference in spleen weight between the control- and DSS-groups was identified. In the DSS+SBG group, however, spleen weight was significantly higher than observed in the DSS group (P=0.046, DSS+SBG vs. DSS) (FIG. 6 a and Table 3). Although not statistically significant, the SBG group appeared to have a slightly higher spleen weight compared to control mice.

Furthermore, oral SBG administration appeared to limit colitis-associated thymus involution (P=0.042, DSS+SBG vs. DSS) (FIG. 6 b and Table 3).

TABLE 3
Data summary

| Parameter | | | | |
|---|---|---|---|---|
| Median and | Experimental groups | | | |
| (range) | Control | SBG | DSS | DSS + SBG |
| Histopat. | 1.5 (0-3) | 1.5 (0-3) | 14.5 (6-20) | 10 (3-17) |
| score | | | | |
| Colon | 91.5 (79-119) | 88.5 (72-103) | 67.5 (55-76) | 73 (60-85) |
| length(mm) | | | | |
| Spleen | 99.6 (93.1-117.2) | 106.2 (90-131.7) | 88.2 (34.7-144.1) | 120 (40.5-148) |
| weight(mg) | | | | |
| Thymus | 15.5 (9.3-26.3) | 15.95 (12.5-26) | 9.4 (3.4-26) | 12.4 (8.3-25.2) |
| weight (mg) | | | | |

### Oral SBG Administration Limits Systemic Inflammation in Acute Colitis.

To investigate whether oral SBG administration could have an impact on systemic inflammation, the levels of various inflammatory mediators were determined in the four experimental groups of mice post-sacrifice.

As can be seen in FIG. 8, the levels of TNFα, IFNγ, IL-1α, IL-1β, IL-2, IL-3, IL-5, IL-6, IL-10, IL-13, IL-17, GM-CSF, MCP-1 and MIP-1β were all increased in the DSS mice as compared to the control group. Levels of these inflammatory mediators were however reduced in the DSS+SBG group as compared to the DSS group, indicating that SBG can reduce systemic inflammation in acute colitis by reducing levels of these inflammatory mediators, which are increased in association with the disease.

No overall difference in the levels of IL-4, IL-9, IL-12p40, IL-12p70, Eotaxin, Rantes, KC, MIP-1α and G-CSF was observed between the DSS and DSS+SBG groups.

### 1.4 Conclusion:

Example 1 clearly demonstrates a beneficial effect of oral SBG administration on DSS-induced experimental IBD:
1) Oral SBG treatment reduces colitis-associated weight loss in experimental animals.
2) Oral SBG treatment reduces colitis-associated mortality in experimental IBD.
3) Oral SBG treatment reduces colonic inflammation and tissue damage in experimental colitis
4) Oral SBG treatment reduces colitis-associated colon shortening in experimental colitis.
5) Oral SBG treatment limits colitis-associated thymus involution.
6) Oral SBG treatment limits systemic inflammation in acute colitis.

### 1.5 Additional Experimental Detail:

### Effect of Oral SBG Administration on Weight Loss in Acute Colitis.

Mice were pretreated with SBG or regular drinking water for 7 days, prior to induction of acute colitis by oral exposure to DSS for 7 days. Body weight was recorded daily during, pretreatment, colitis induction and for 4 subsequent days during the acute- and initial recovery phase (Acu/rec), after which the animals were sacrificed. Control animals (Ctr, n=12) received regular drinking water throughout the experiment. SBG treated animals (SBG, n=12) received SBG-supplemented drinking water (100 mg/mL) throughout the experiment. DSS treated animals (DSS, n=16) received regular drinking water in the pretreatment phase, DSS-supplemented drinking water (1.5% w/v) in the induction phase, and regular drinking water in the acute-/recovery phase. DSS and SBG combination treated animals (DSS+SBG, n=15) received SBG-supplemented drinking water in the pretreatment phase, combined DSS/SBG-supplemented drinking water in the induction phase, and SBG-supplemented drinking water in the acute-/recovery phase. Body weight is expressed as percentage of base line (BL) values, mean±SEM. E=Euthanized mice: body weight reduction >20% or moribund. *P<0.05, **P<0.01, ***P<0.001, DSS versus DSS+SBG as determined by two-way analysis of variance with Bonferroni posttest. Data presented are pooled from two independent experiments.

### Effect of Oral SBG Administration on Survival in Acute Colitis.

Mice were pretreated with SBG or regular drinking water prior to induction of acute colitis by oral exposure to DSS as described. Mortality/forced euthanasia was recorded in the 4-day acute-/recovery phase following DSS-removal and expressed as percent of the initial group size. Humane endpoint criterion was body weight loss >20% of baseline weight. Unmistakably moribund animals not meeting the weight loss criterion were also euthanized. Data presented are pooled from two independent experiments.

### Effect of Oral SBG Administration on Colonic Inflammation and Tissue Damage in Acute Colitis.

Mice were pretreated with SBG or regular drinking water prior to induction of acute colitis by oral exposure to DSS as described. Postmortem, colons were excised, flushed with PBS and prepared for histological analysis. Formalin fixed, paraffin embedded, H&E stained sections were examined for inflammatory cell infiltration, tissue damage, absence or presence of lymphoid aggregates and epithelial regeneration (Table 2). A) Representative distal colon H&E sections from control animals (Ctr, top left), SBG treated animals (SBG, top right), DSS treated animals (DSS, bottom left) and DSS and SBG combination treated animals (DSS+SBG, bottom, right), original magnification 100×. B) Proximal, medial and distal colon segments were evaluated and an overall histopathology score was calculated for each mouse. Open symbols indicates animals euthanized for animal welfare reasons prior to the scheduled end point. Bars represent median values. Data presented are pooled from two independent experiments.

### Effect of Oral SBG Administration on Colitis-Associated Colon Shortening.

Mice were pretreated with SBG or regular drinking water prior to induction of acute colitis by oral exposure to DSS as described. Postmortem, colons were excised and the colon length was measured. A) Representative pictures of colons excised from control animals (Ctr), SBG treated animals (SBG), DSS treated animals (DSS) and DSS and SBG combination treated animals (DSS+SBG). Images have been digitally enhanced (Adobe Photoshop CS 8.0, Adobe Systems Inc., San Jose, Calif., USA). B) Colon length in mm. Open symbols indicates animals euthanized for animal welfare reasons prior to the scheduled end point. Bars represent median values. Data presented are pooled from two independent experiments.

Effect of Oral SBG Administration on Spleen and Thymus Weight in Acute Colitis.

Mice were pretreated with SBG or regular drinking water prior to induction of acute colitis by oral exposure to DSS as described. Postmortem, spleen (A) and thymus (B) were excised. Following formalin fixation, organ weight (mg) was recorded. Open symbols indicates animals euthanized for animal welfare reasons prior to the scheduled end point. Bars represent median values. Data presented are pooled from two independent experiments.

### Example 2

### 2.1 Experimental Design

The effect of the feed β-1,3/1,6-glucan product MacroGard® and the hydrolysed yeast cell/whole yeast cell product PatoGard™ were tested with animal feed products with an unconventionally high concentration of plant proteins. The purpose of the high-protein feed was to generate conditions in the intestines and to evaluate the effect of the products MacroGard® and PatoGard™ in the prevention and treatment of such conditions. The fish used in these trials was atlantic salmon (Salmo salar).

A high content of plant proteins over 15% leads generally to reduced colon health in fish as fish are not used to this kind of high protein diet. In this trial the total protein content was increased to 32% thus leading to detrimental effects on the fish colon. For purpose of investigating the effects of different plant proteins, both soy and sunflower proteins were used and compared to the golden standard being an easy to digest fish meal product.

The trial included in total nine different groups. The different groups were fed with PatoGard™ and MacroGard® (65%, β-1,3/1,6-glucan). The control groups received only the prepared high-plant protein containing animal feed. The distribution can be seen in Table 4 below.

Feed 1 FM: Feed with fishmeal only
Feed 2: FMS: Feed with fish meal and 32% soy
Feed 3: FMSPG: Group 2 feed with 2000 mg PatoGard™
Feed 4: FMSMG: Group 2 feed with 1000 mg MacroGard®
Feed 5: FMSS: Feed with fish meal, 15% soy and 15% sunflower meal
Feed 6: FMSSPG: Group 5 feed with 2000 mg PatoGard™
Feed 7: FMSSMG: Group 5 feed with 1000 mg MacroGard®

The seven groups consisted of 150 fish which were bred in 5×5×5 meter trail basins in the sea. The groups were fed with the respective feeds for 71 days. After that period the fish were measured and weighed and tissue samples were taken from the intestines of 27 randomly chosen fish. Tissue alterations were registered by using a standard method and classified after the Urán-score. The score focuses on (1) the presence and size of supranuclear vacuoles (2) degree of widening of the lamina propria of simple folds (3) amount of connective tissue between the base folds and stratus compactum of the and (4) degree of thickening of the mucosal folds. Every check point is classified on a scale from 1-5 where 1 means undamaged and 5 is a lethal damage.

### 2.2 Results and discussion

TABLE 4
Intestinal health with group 1-7 feed measured
by using the Uran score

| Feed | Defintion | Uran- |
|---|---|---|
| group | feed | score |
| 2 | FMS | 3.68 |
| 3 | FMSMG | 3.43 |
| 4 | FMSPG | 3.37 |
| 5 | FMSS | 2.05 |
| 6 | FMSSMG | 1.61 |
| 7 | FMSSPG | 1.23 |
| 1 | FM | 1.13 |

Detrimental intestinal health in group 2 and 5 fish feed was considerably reduced by adding Patogard™ and MacroGar® to the feed (groups 3, 4, 6 and 7). The addition of Patogard™ gave a significant reduction in relation to the feeds including soy alone or a combination of soy and sunflower. These results show clearly that both Patogard™ and MacroGard® eliminate the detrimental effect of plant proteins in the feed and lead thus to an improved bowel health.

### Example 3

### 3.1 Ingredients and diets

The formulation and composition of the diets is given in Tables 5 and 6, respectively. A standard fish meal based control diet (FM), a high-vegetable diet with 13.2% extracted and toasted soybean meal [SBM] and 13.5% extracted sunflower meal [SFM] (FM+SS), and a high-vegetable diet with 29.9% soybean meal (FM+S) were manufactured by high-pressure moist extrusion by Skretting (Averøy, Norway). The particle size was 6 mm, and all diets were dried prior to coating with fish oil.

Prior to coating with oil, batches of the basis FM+SS diet was first coated with 1000 mg of MacroGard® (FM+SS+1000MG) or 2000 mg PatoGard® (FM+SS+2000PG) per kg diet. Likewise, batches of the basis FM+S diets was pre-coated 500 (FM+S+500MG) or 1000 (FM+S+1000MG) mg MacroGard® or 1000 (FM+S+1000PG) or 2000 (FM+S+2000PG) PatoGard® per kg diet. This gave a series of nine experimental diets.

TABLE 5
Formulation of the experimental diets.

| | | | |
|---|---|---|---|
| | | FM + SBM + SFM | |
| Diet code | FM-control | (SS) | FM + SBM (S) |

Formula, g kg⁻¹

| | | | |
|---|---|---|---|
| LT-fish meal | 525.0 | 300.0 | 242.0 |
| SBM | | 135.0 | 320.0 |
| SFM | | 135.0 | |
| Wheat gluten | 0.0 | | 10.0 |
| Wheat | 188.0 | 116.5 | 100.5 |
| Fish oil | 286.0 | 291.0 | 305.0 |
| Lysine | | 1.0 | 1.0 |
| Methionine | | 1.5 | 1.5 |
| MCP* | 1.0 | 20.0 | 20.0 |

*Mono Calcium Phosphate

TABLE 6
Composition of the experimental diets

| Basic diet | FM | FM + S | FM + SS | FM + S | | FM + SS | | | |
|---|---|---|---|---|---|---|---|---|---|
| Added | MG | PG | MG | PG | MG | PG | | | |
| Dose, | 500 | 1000 | 1000 | 2000 | 1000 | 2000 | | | |
| mg kg⁻¹ | | | | | | | | | |
| Dry | 937.3 | 934.0 | 941.0 | 933.1 | 930.3 | 932.7 | 930.8 | 940.8 | 942.7 |
| matter, g | | | | | | | | | |
| Crude | 385.0 | 348.5 | 340.9 | 347.3 | 346.0 | 351.0 | 345.8 | 341.2 | 359.5 |
| protein*, g | | | | | | | | | |
| Lipid, g | 336.7 | 327.6 | 347.6 | 335.0 | 319.6 | 331.9 | 326.1 | 348.6 | 352.5 |
| Starch, g | 100.6 | 53.9 | 57.1 | 58.0 | 53.7 | 59.6 | 54.2 | 56.7 | 71.3 |
| Ash, g | 80.0 | 62.8 | 68.6 | 63.2 | 59.7 | 64.9 | 62.6 | 69.1 | 73.4 |
| Energy, MJ | 24.3 | 24.2 | 24.7 | 24.4 | 24.2 | 24.1 | 24.0 | 24.6 | 24.9 |

*CP; N × 6.25

### 3.2 Fish, Rearing Conditions, and Sampling

Atlantic salmon (Salmo salar) were fed the experimental diets for a total of 69 to 71 feeding days. Prior to the experiment, the fish were fed commercial diets (Skretting AS, Stavanger, Norway). The experiment was initiated in week 25 and terminated in week 36 of 2006. The water temperature varied from 12.3 to 17.4° C. during the course of the experiment, averaging 15.3° C.

At the start of the experiment, 27 groups of salmon (679 g, 150 fish per group) were randomly distributed to 5×5×5 m³ sea pens. Each diet was then allocated to three groups of fish in a triplicate randomised experimental design. The fish were continuously fed by electrically driven feeders, and uneaten feed was collected from underneath the pens and pumped up into wire mesh strainers as described by Einen (1999). The feeding rate was planned to be 15% in excess, and was adjusted according to the recorded overfeeding every three days as described by Helland et al. (1996).

The fish were weighed in bulk at the start of the experiment and on feeding day 70. At the final weighing a sufficient number of fish were also anesthetised with tricaine methanesulfonate (MS 222, Argent Chemical Laboratories Inc., Redmont, Wa, USA) and stripped as described by Austreng (1978) to collect faeces for digestibility estimation. The faecal samples were pooled per pen and immediately frozen at −20° C.

### 3.3 Chemical Analyses and Histological Examination

Faeces were freeze-dried prior to analyses. Diets, and freeze dried faeces were analysed for dry matter, ash, nitrogen, lipid, starch (determined as glucose after hydrolysis by α-amylase and amylo-glucosidase, followed by glucose determination by the “GODPOD method” (Megazyme, Bray, Ireland)), gross energy (Parr 1271 Bomb calorimeter, Parr, Moline, IL, USA) and yttrium (at Jordforsk, Ås, Norway, by inductivity coupled plasma (ICP) mass-spectroscopy, as previously described by Refstie et al. (1997)).

### 3.4 Statistical Analyses

The results were analysed by the General Linear Model procedure in the SAS computer software (SAS, 1985). Mean results per pen were subjected to one-way analysis of variance (ANOVA) with diet as the independent variable. Significant differences were indicated by Duncan's multiple range test. The level of significance was P0.05, and the results are presented as mean±s.e.m. (standard error of the mean).

### 3.5 Results and Discussion

Feeding the FM+S and FM+SS diets generally resulted in lower dry matter content (i.e. more water) in the faeces, than when feeding the FM diet, indicating diarrhoea (Table 7 below).

TABLE 7
Apparent digestibility of nutrients and retention of nitrogen and energy
by the fish after feeding the experimental diets for 70 days (n = 3).

| | Faecal dry | Apparent digestibility (%) of | | | | Retention (%) of | |
|---|---|---|---|---|---|---|---|
| Diet | matter, % | Nitrogen | Lipid | Starch | Energy | Nitrogen | Energy |
| FM | 12.2^{a} | 71.0 | 84.5^{ab} | 27.5^{b} | 68.7 | 46.5^{c} | 58.9 |
| FM + S | 8.8^{c} | 75.1 | 77.3^{bc} | 49.8^{a} | 64.9 | 40.4^{d} | 45.5 |
| FM + SS | 8.7^{c} | 75.6 | 87.7^{a} | 46.1^{a} | 70.8 | 50.1^{b} | 55.1 |
| FM + S + 1000 MG | 8.5^{c} | 73.3 | 70.0^{c} | 51.0^{a} | 59.3 | 39.8^{d} | 46.1 |
| FM + S + 2000 PG | 8.8^{c} | 72.8 | 71.0^{c} | 47.3^{a} | 59.9 | 38.9^{d} | 44.8 |
| FM + SS + 1000 MG | 9.5^{bc} | 77.5 | 88.2^{a} | 48.6^{a} | 71.5 | 47.8^{bc} | 55.1 |
| FM + SS + 2000 PG | 10.7^{b} | 77.1 | 89.7^{a} | 42.6^{a} | | 55.5^{a} | |
| ANOVA: | | | | | | | |
| P | <0.0001 | 0.27 | 0.0008 | 0.02 | | <0.0001 | |
| \sqrt{MSE} | 0.7 | 3.4 | 5.2 | 7.2 | | 1.7 | |

Different superscripts ^{abcd} within column indicates significant differences as indicated by Duncan's Multiple Range Test (P < 0.05).

When comparing the FM+SS diets, this was significantly ameliorated when adding 2000 mg kg-1 of PG, and the diarrhoeic condition also tended to improve when adding 1000 mg kg-1 of MG. No such effect of MG and PG was seen when added to the FM+S basis diet.

### Example 4

### Effect of Soluble Beta-Glucan on the Gut and Gut-Associated Lymphoid Tissue in Mice

### 4.1 Experimental Design:

A model was established to evaluate the effects of soluble glucans (here the product SBG from the company Biotec Pharmacon ASA) on the gut and gut-associated lymphoid tissue (GALT) and the intestinal epithelium of healthy mice. The outcome was assessed by studying body weight changes, fluid consumption, the number and lymphocyte composition of Peyer's patches (PPs), the cross section area and lymphocyte composition of mesenteric lymph nodes (MLNs) and the number and distribution of Ki67-positive cells and goblet cells. The number of macroscopically visible PPs was determined by visual inspection of the excised intestine. The cross section area of formalin fixed MLNs was determined by analysis of hematoxylin and eosin (H & E) stained sections. The composition of major lymphocyte subsets in the PPs and MLNs was examined by means of flow cytometry. Proliferating, Ki67-positive, intestinal epithelial cells were identified by immunohistochemistry.

Male BALB/c mice were maintained in the minimal disease unit at the Centre for Comparative Medicine at Rikshospitalet University Hospital, Oslo, Norway for at least one week before they were entered into experiments. Animals were housed 2 mice per cage, supplied with water and conventionally fed ad libitum. Cages were kept at 21±1° C. and 55±10% relative humidity. Light conditions consisted of alternating 12h light/dark cycles with one hour dusk and dawn.

Mice were randomly distributed into two experimental groups; SBG treated mice and control (Ctr) mice receiving SBG-supplemented water (100 mg/L) or regular drinking water, respectively, ad libitum for 20 days (0-19). Body weight and fluid consumption was recorded and mice were monitored for clinical signs of morbidity throughout the experiment.

Three identical, but separate, experiments were performed. Tissue samples conserved for subsequent histological investigation were collected in the two first experiments, whereas fresh material for flow cytometric analysis was collected in the third experiment.

Animals were anesthetized by subcutaneous injection of Hypnorn® and midazolam (50-75 μL/10g body weight) prior to cardiac puncture. Postmortem mice were soaked in 70% ethanol and fixed to a dissection board. The abdomen was opened and the, MLNs, inguinal lymph nodes (ILNs), spleen and intestine were excised. The small intestine was examined for macroscopically visible PPs and identified PPs were excised. The colon was flushed with cold PBS to remove fecal contents prior to fixation. Tissue samples collected for subsequent histological analysis were kept on ice and fixed in 10% formalin for 24 h at 4° C. Fixed tissue samples were transferred to PBS with 0.1% formalin and stored at 4° C. for subsequent preparation and analysis. PPs, MLNs, ILNs and spleens isolated for subsequent flow cytometric analysis were transferred to ice cold FM. Blood collected by cardiac puncture, in lithium heparin vacuum tubes, was kept on ice until subsequent analysis.

### Histochemistry and Immunohistochemisty

Formalin fixed biopsies were processed using an automated tissue processor and subsequently embedded in paraffin. Sections were cut at 4 μm and placed on polysine coated slides. Microscopy and image analysis was performed by an examiner blinded to the sample identity.

### MLN Cross Section Area

MLN sections were manually stained with hematoxylin and eosin (H&E) and examined in a light microscope fitted with a digital camera and imaging software. The MLN cross section area was calculated by analyzing microphotographs using a build in feature in the microscope imaging software. Briefly, the perimeter of the MLN section was marked using an interpolating drawing tool, and the area was calculated based on the number of pixels included.

### Goblet Cell Count

Sections from the distal colon were deparafinized in xylene and ethanol and rehydrated in distilled water before staining with hematoxylin, alcian blue and periodic acid Schiff reagent in an automated tissue stainer. Colonic sections were examined in a light microscope. The number of goblet cells was determined by counting AB/PAS positive cells in 20 well oriented crypts, displaying the intact crypt height, and expressed as the mean number of positive cells per crypt. Intra crypt distribution of goblet cells was indicated as the number of positive cells in the basal-, central- and top ⅓ of the crypt. Illustration microphotographs were acquired using a light microscope fitted with a camera.

### Immunohistochemistry

Formalin fixed sections from distal colon biopsies were deparafinized in xylene and ethanol, rehydrated in PBS; and boiled in CA antigen retrieval buffer for 20 minutes. Sections were incubated with primary antibodies or concentration- and isotype-matched control antibodies over night at 4° C. Following washing in PBS, sections were incubated with fluorochrome-conjugated secondary antibody for 3 h at room temperature. Nuclei were stained with Hoechst stain. Sections were examined in a fluorescence microscope fitted with a digital camera and imaging software.

The number of proliferating Ki67 positive epithelial cells and the size of the proliferative zone were determined by analysis of digital images. Areas of the section displaying intact crypt height were chosen for analysis. Cell count was expressed as the mean number of positive cells per crypt, counting ≧8 crypts, and the proliferative zone was expressed as a percentage of the total crypt height.

IEL numbers were determined by counting CD3 positive cells clearly located within the epithelium. The entire circumference of a colon section was screened directly in the fluorescence microscope.

### Flow Cytometry

Spleens, MLNs, ILNs and PPs were disrupted and ground between two sheaths of nylon mesh in FM buffer using flat spatula-tip tweezers. The homogenate was filtrated over a fresh nylon mesh, centrifuged (1400 rpm/410 g, at 4° C. for 4 min) and washed in FM to produce single cell suspensions. 1 million MLN-, ILN- and PP cells and 300 μL whole blood were incubated with 100 μL of a staining cocktail, consisting of antibodies and 0.1 mg rat IgG per 100 μL in FM buffer, for 30 min on ice in the dark. Cells were washed in FM, centrifuged as described above and incubated with APC-Cy7 conjugated streptavidin for 20 min on ice in the dark to label the biotinylated antibody employed. Following washing in FM, tissue-derived single cells were resuspended in paraformaldehyde (1% in PBS) and incubated for 5 min on ice in the dark for fixation. The fixative was removed and cells were resuspended in FM and stored in the dark at 4° C. for subsequent flow cytometry analysis. Leukocytes were prepared for analysis from the whole blood staining reaction by lysis of erythrocytes in OptiLyse B according to manufacturer's instructions. The lysis solution was removed by centrifugation, cells were fixed, resuspended in FM, and stored for analysis as described above. Unstained spleen, MLN, ILN, PP and OptiLyse B treated whole blood cells served as controls. Cell suspensions were analysed on a flow cytometer.

### Statistical Analysis

Body weight and fluid consumption data were expressed as mean values with standard deviation of the mean (SD) and analyzed using two-way analysis of variance (ANOVA) with Bonferroni post test. PP number, MLN cross section area, goblet cell numbers, epithelial proliferation and IEL numbers were expressed as median values and analyzed using the Mann-Whitney test. Flow cytometry data on lymphocyte composition was expressed as mean values with standard deviation (SD) and analyzed using the Mann-Whitney test. Highly suspect outlier values, unlikely to represent random sampling from a Gaussian population, were identified by Grubb's outlier detection test and excluded from further analysis. All statistical analysis was carried out using GraphPad Prism, version 4 (GraphPad Software, San Diego, Calif., USA). Differences at P<0.05 were considered statistically significant.

### 4.2 Results:

### Effect of SBG Supplementation on Body Weight and Fluid Consumption

Male BALB/c mice were randomly distributed into two experimental groups: A group receiving SBG-supplemented drinking water (SBG) and a control group receiving pure drinking water (Ctr). To monitor the overall health condition of the experimental animals in response to oral SBG administration, body weight was recorded. The mice steadily gained weight and no difference in body weight dynamics between Ctr and SBG treated animals was observed (FIG. 9). SBG appeared to be well tolerated and no clinical signs of morbidity were noted.

To further investigate the effect of SBG supplementation on appetite and overall activity, and importantly to estimate the daily and total SBG dose acquired, the average fluid consumption per mouse was calculated. Fluid consumption was approximately 4-7 ml/mouse/day, corresponding to a daily (3-glucan dose of 15-30 mg/kg body weight in the SBG group. No difference in fluid consumption between the experimental groups was recorded (FIG. 9).

### Oral SBG Administration Affect Mucosal Inductive Sites

To investigate the effect of oral SBG administration on GALT, PPs and MLNs, essential mucosal inductive- and regulatory sites, were examined. In the SBG group, the median number of macroscopically observable PP in the small intestine was approximately 40% higher than what we observed in the Ctr group (P<0.01) (FIG. 10 A). Furthermore, we identified a significant increase in the MLN size in SBG-treated mice. In the SBG group, the median cross section area of isolated MLNs was approximately 35% larger than what we observed in the Ctr group (P<0.05) (FIG. 10 B). Assuming sphereoid LNs, this corresponds to an estimated volume increase of 50-60%.

Despite the evident changes in GALT following oral SBG administration, characterization of the major lymphocyte populations (CD4^{pos}, CD8^{pos}, CD19^{pos} cells) revealed no differences between the experimental groups neither for the PPs nor the MLNs (FIG. 11). Similarly, lymphocyte composition in blood leukocytes, spleen- and ILN single cell suspensions, representing the systemic compartment, was not altered by oral SBG administration (data not shown). Flow cytometry analysis revealed the presence of Dectin-1^{pos} cells, primarily MHC class II^{Pos}, CD11b^{pos} or CD11c^{pos} cells, i.e. macrophages and dendritic cells (DCs), in all cell preparations examined. However, oral SBG administration did not appear to change the expression profile of this β-glucan receptor. Of MLN single cells isolated from SBG treated mice, 3.5±1.8% were Dectin-1 positive vs. 3.3±1.2% in Ctr animals (mean±SD). The corresponding numbers for PP were 0.22±0.04% vs. 0.23±0.05%, for ILN 3.2±1.2% vs. 3.4±0.7%, for spleen 6.5±1.6% vs. 7.1±1.2%, and for blood leukocytes 7.9±1.6% vs. 8.0±1.9%.

### Oral SBG Administration Increase Epithelial Proliferation

To examine natural defense functions mediated by the different intestinal epithelial cell types, we first analyzed the number and distribution of mucus producing goblet cells. Oral SBG administration did not affect the number or intra crypt distribution of goblet cells in the distal colon. The number of AB/PAS positive goblet cell per crypt in SBG treated mice was 6.6[3.1-8.8] compared to 7.6[5.4-10.3] in the Ctr group (mean and [range])(FIG. 13). We also stained sections for IELs, but very few IELs were identified in the distal colon sections and no difference in IEL numbers between SBG treated mice and controls was revealed. The number of CD3^{pos} IELs per section in SBG treated mice was 12.4[5.0-21.0] compared to 12.3[6.5-21.5] in the Ctr group (mean and [range]).

Next, we investigated the effect of oral SBG administration on the intestinal epithelium, a mucosal effector site. In mice treated with SBG the number of proliferating epithelial cells in the distal colon was significantly higher than what we observed in control animals (FIG. 12). The median number of Ki67^{pos} cells per crypt in the SBG group was 37% higher than in the Ctr group (P<0.01). Also, the median size of the proliferative zone was 25% larger in the SBG group (P<0.001) compared to controls.

### 4.3 Conclusion:

This Example clearly demonstrates that administration of SBG has an effect on PPs and MLNs, vital mucosal inductive sites. PPs and MLNs play a central role in induction and maintenance of oral tolerance and systemic ignorance to the intestinal microbiota.

It is demonstrated here for the first time that orally administered SBG stimulate GALT and epithelium, vital inductive- and effector sites of the mucosal immune system, respectively. It is demonstrated that oral administration of soluble β-glucan has an effect on GALT (PP and MLN) size. Although speculative, it is plausible that β-glucan-laden cells migrating from the intestinal epithelium to GALT may contribute to the observed expansion of MLNs and PPs.

The relative content of CD4^{pos} and CD8^{pos} T cells and CD19^{pos} B cells, major lymphocyte populations, of MLNs and PPs by flow cytometry have been characterized and no significant difference found between SBG treated mice and controls. We observed no change in Dectin-1 expression in response to oral SBG administration. Oral SBG administration did not change the number and distribution of goblet cells in the colon.

Here we report that SBG increased the number of proliferating epithelial cells as well as the size of the proliferative zone in the colon when administered orally. Data presented here indicates that the protective effect of SBG in experimental colitis is, in part, due to stimulatory effects on epithelial proliferation and thus conceivably on epithelial barrier restitution and function. Thus we report that oral β-glucan administration stimulates intestinal epithelial proliferation.

We demonstrate that oral administration of SBG, a Saccharomyces cerevisiae-derived water-soluble β-glucan, stimulated formation and/or expansion of PPs and MLNs. Furthermore, SBG stimulated proliferation of mucosal epithelial cells, suggesting that SBG may also affect intestinal barrier function. The data suggests that β-glucans enhance host protection, in part, by effects on the mucosal immune system. The stimulatory effects may be mediated both on the mucosal inductive sites of immune responses as well as the effector sites of immune defense.

1-21. (canceled)

22. A method of treatment or prevention of inflammatory bowel disease or a related disease of abnormal bowel function in an animal in need thereof which comprises administering to said animal an effective amount of a glucan derived from yeast having a beta-(1,3)-backbone with one or more beta-(1,3)-side chains linked thereto.

23. The method of claim 22 wherein the yeast is Saccharomyces cerevisiae.

24. The method of clam 22 wherein the glucan is at least partially isolated from other cell wall components.

25. The method of claim 22 wherein the glucan has a chemical structure which is altered as compared to its naturally occurring structure.

26. The method of claim 22 wherein the glucan is underivatized by chemical groups.

27. The method of claim 22 wherein the glucan is essentially free of repetitive beta-(1,6)-linked glucosyl units.

28. The method of claim 22 wherein the glucan is in particulate form.

29. The method of claim 22 wherein the glucan is soluble.

30. The method of claim 28 wherein the molecular weight of the glucan is in the range of 150 kDa to 300 kDa.

31. The method of claim 29 wherein the molecular weight of the glucan is in the range of about 6 kDa to 30 kDa.

32. The method of claim 22 wherein the glucan is administered orally.

33. The method of claim 32 wherein the glucan is administered in a dose range of 0.1 to 500 mg per kg body weight per day.

34. A method of treatment or prevention of inflammatory bowel disease or a related disease of abnormal bowel function in an animal in need thereof which comprises administering to the animal an effective amount of a meal derived from Asteraceae.

35. A method of treatment or prevention of inflammatory bowel disease or a related disease of abnormal bowel function in an animal in need thereof which comprises administering to the animal an effective amount of a protein derived from Asteraceae.

36. The method of claim 34 or 35 wherein the Asteraceae is from the genus Helianthus.

37. The method of claim 36 wherein the Asteraceae is Helianthus annus.

38. The method of claim 22, 34 or 35 wherein the inflammatory bowel disease is ulcerative colitis or Crohn's disease.

39. The method of claim 22, 34 or 35, wherein the related disease of abnormal bowel function is selected from the group consisting of constipation, diarrhea, fecal incontinence and conditions and diseases resulting due to surgery.

40. The method of claim 22 wherein the glucan is administered with a second active agent for the treatment of inflammatory bowel disease or a related disease of abnormal bowel function, wherein the glucan and second active agent are administered simultaneously, separately or sequentially.

41. The method of claim 34 wherein the meal is administered with a second active agent for the treatment of inflammatory bowel disease or a related disease of abnormal bowel function, wherein the glucan and second active agent are administered simultaneously, separately or sequentially.

42. The method of claim 35 wherein the protein is administered with a second active agent for the treatment of inflammatory bowel disease or a related disease of abnormal bowel function, wherein the glucan and second active agent are administered simultaneously, separately or sequentially.

43. A kit or administration device comprising
a) a glucan derived from yeast having a beta-(1,3)-backbone with at least one beta-(1,3)-side chain linked thereto, a meal derived from Asteraceae or a protein derived from Asteraceae and
b) information material which describes administering said glucan, meal or protein to a human or other animal for the treatment or prevention of inflammatory bowel disease or a related disease of abnormal bowel function.
